(19) United States
(12) Patent Application Publication
(10) Pub. No.: US 2026/0144589 A1
(43) Pub. Date: May 28, 2026

(54) ABLATION DEVICES, SYSTEMS, AND METHODS

(71) Applicant: Field Medical, Inc., Cardiff By The Sea, CA (US)

(72) Inventors: Steven Richard MICKELSEN, Cardiff By The Sea, CA (US); Joseph P. HIGGINS, Wayzata, MN (US); Stephen A. LEEFLANG, Sandy, UT (US); David Colton JACKSON, Cottonwood Heights, UT (US)

(21) Appl. No.: 19/397,849

(22) Filed: Nov. 21, 2025

Related U.S. Application Data
(60) Provisional application No. 63/723,697, filed on Nov. 22, 2024.
(60) Provisional application No. 63/725,940, filed on Nov. 27, 2024.
(60) Provisional application No. 63/838,409, filed on Jul. 3, 2025.
(60) Provisional application No. 63/895,948, filed on Oct. 8, 2025.
(60) Provisional application No. 63/900,483, filed on Oct. 16, 2025.

(51) Int. Cl. A61B 18/14; A61B 18/00

(52) U.S. Cl. CPC A61B 18/1492; A61B 2018/00029; A61B 2018/00351; A61B 2018/00577; A61B 2018/00613; A61B 2018/1405; A61B 2018/1467; A61B 2218/002

(57) ABSTRACT

An ablation catheter is disclosed, comprising an elongate body having a fluid lumen extending therethrough. The catheter includes a distal electrode assembly featuring a central insert electrode and an outer electrode that surrounds it at the distal end. The insert electrode has a non-circular cross-sectional shape. Fluid passages are formed between portions of the insert electrode and an inner surface of the outer electrode. The fluid passages are in fluid communication with the lumen and are configured to deliver fluid to a tissue-contacting surface of the catheter, enhancing ablation safety and efficiency. Also disclosed are related systems and methods of using the catheter for therapeutic ablation.

### CROSS REFERENCE TO RELATED APPLICATIONS

This application claims priority to U.S. Provisional Patent Application No. 63/723,697 filed Nov. 22, 2024, U.S. Provisional Patent Application No. 63/725,940 filed Nov. 27, 2024, U.S. Provisional Application No. 63/838,409 filed Jul. 3, 2025, U.S. Provisional Application No. 63/895,948 filed Oct. 8, 2025, and U.S. Provisional Application No. 63/900,483 filed Oct. 16, 2025, the content of each of which is incorporated herein by reference in its entirety for all purposes.

### FIELD

The systems, devices, and methods described herein relate to ablation catheters for treating tissue. The ablation catheters may deliver energy to the tissue as a pulsed electric field. The delivered energy may be used to modify tissue, e.g., cardiac tissue, to treat arrhythmias such as atrial fibrillation and ventricular tachycardia.

### BACKGROUND

Ablation catheters are widely used in a variety of medical procedures to deliver electrical energy to target tissue. In cardiac applications, for example, ablation catheters may be introduced into the heart to affect, isolate, or otherwise modify tissue regions such as the pulmonary veins or myocardial tissue. Modifying these tissues can interrupt conduction pathways associated with arrhythmias, including atrial fibrillation and ventricular tachycardia, and can support restoration or maintenance of normal cardiac rhythm.

Certain treatment modalities use high-voltage, short-duration electrical pulses to create an electrical field adjacent to the target tissue. These pulses can induce electroporation—either reversible or irreversible—by altering the permeability of cellular membranes. Irreversible electroporation may allow targeted modification of tissue with reduced thermal injury compared to traditional radiofrequency ablation. Because these procedures involve delivery of substantial electrical energy, many systems employ irrigation to cool the tissue and the catheter tip, to control lesion characteristics, and to limit thermal buildup or coagulum formation.

Various catheter designs have been developed to deliver energy and irrigation fluid to the distal region of the device. Some known systems include an electrode at the catheter tip and a separate irrigation lumen positioned near the electrode to provide cooling fluid. However, providing multiple structures—such as separate electrode components, separate fluid lumens, and interconnecting features—may increase the overall catheter size and complexity. Integration of fluid and electrical pathways into a more compact structure remains an area of ongoing development.

In addition, a number of catheters utilize bipolar configurations with a tip body electrode and one or more proximal ring electrodes positioned along the catheter shaft. These bipolar arrangements can establish an electric potential between electrodes spaced relatively close together. While effective for many applications, some known bipolar geometries may yield non-uniform or asymmetric electric field distributions around the catheter tip. For example, in some configurations, the electric field may penetrate target tissue more deeply along some orientations than others, or may exhibit reduced field intensity at the distal most end of the catheter. As a result, the therapeutic depth or contour of the lesion may vary depending on catheter positioning.

Other approaches rely on unipolar configurations, in which a relatively small intracardiac electrode is paired with a large surface patch electrode positioned extracorporeally. Although unipolar approaches may alter the spatial distribution of the electric field, they may also introduce different trade-offs. For example, unipolar arrangements may generate broader field spreads or reduced peak electric field strengths near the catheter tip compared to closely spaced bipolar configurations.

Accordingly, there remains an ongoing need for improved ablation catheters and methods that can efficiently deliver electrical energy to tissue, while managing irrigation flow and controlling fluid dynamics, reducing device complexity or size, and supporting more uniform or radially symmetric electrical field distributions in the region surrounding the catheter tip. Improved catheter structures that integrate conductive elements and fluid pathways within a compact distal assembly may address one or more of these needs.

### SUMMARY

Catheters may comprise an elongate body having a fluid lumen extending therethrough and a distal electrode assembly coupled to the elongate body. The distal electrode assembly may comprise an insert electrode and an outer electrode surrounding at least a portion of the insert electrode and electrically coupled thereto. One or more interstitial spaces between the insert electrode and the outer electrode may define one or more fluid passages in fluid communication with the fluid lumen. The one or more fluid passages may be configured to provide laminar fluid flow through the distal electrode assembly.

The outer electrode may surround a proximal portion of the insert electrode disposed within a lumen in a tip of the catheter. The one or more fluid passages may comprise a length of about 1.5 mm to about 3 mm. A ratio of a maximum transverse dimension of the insert electrode and a length of one or more of the fluid passages may be about 1:4 to about 1:3.

The insert electrode may comprise a non-circular cross-sectional shape. In some variations, the insert electrode may comprise three or more vertices. The insert electrode may comprise a triangular cross-sectional shape. Spaces between the vertices of the insert electrode and an inner surface of the outer electrode may define fluid passages in fluid communication with the fluid lumen. Each vertex may form an interference fit with a corresponding portion of the outer electrode. The vertices of the insert electrode may be spaced at substantially equal angular intervals around a central longitudinal axis of the insert electrode. The insert electrode may further comprise arcuate sidewalls extending between adjacent vertices, wherein each fluid passage may be formed between one of the arcuate sidewalls and an inner surface of the outer electrode. Each arcuate sidewall may be concave. The insert electrode may divide fluid from the fluid lumen into three discrete fluid passages, each comprising an arcuate cross-sectional shape.

Each of the fluid passages may terminate at an outlet positioned around the insert electrode at a distal end surface of the tip of the catheter. At least one outlet may be oriented to direct fluid flow radially outward from a central longitudinal axis of the elongate body. At least one outlet may be oriented to direct fluid radially inward toward a central longitudinal axis of the elongate body. Each fluid passage may extend longitudinally along at least a portion of the insert electrode. Each fluid passage may comprise a width or diameter of about 0.05 mm to about 0.20 mm.

The outer electrode may be positioned at or adjacent to a distal end surface of a tip of the catheter. The outer electrode may comprise a circular cross-sectional shape. The outer electrode may extend proximally into a tip of the catheter. The outer electrode may comprise a length of about 0.5 mm to about 3 mm.

The insert electrode may protrude distally an exposed length beyond a distal end surface of a tip of the catheter. The exposed length of the insert electrode may be about 0.5 mm to about 1 mm. The insert electrode may protrude distally beyond a distal end surface of the outer electrode. The insert electrode may protrude about 0.25 mm to about 3 mm beyond the distal end surface of the outer electrode. The distal end surface of the outer electrode may be substantially flush with a distal end surface of a tip of the catheter. A ratio between the exposed length of the insert electrode and a diameter of the elongate body may be about 1:8 to about 1:4. A ratio between the exposed length of the insert electrode and a maximum transverse dimension of the insert electrode may be configured to promote a substantially symmetric electrical field about a longitudinal axis of the elongate body.

The catheter may further comprise a tip body coupled to a distal end of the elongate body, wherein the distal end surface of the tip of the catheter may be a distal end surface of the tip body. A maximum transverse dimension of the insert electrode may be about 0.25 to about 0.50 times a diameter of the elongate body. The vertices of the insert electrode may converge to form a distal apex. The distal apex may be rounded and configured to contact tissue.

The segments may be coupled by stitching or compression bonding. The plurality of discrete absorbent segments may be radially arranged about the longitudinal axis of the device. The plurality of discrete absorbent segments may be radially dispersed about the central longitudinal axis of the device. A majority of each segment may be decoupled from and independently movable relative to adjacent segments.

The device may be configured to transition between a compressed configuration and at least one expanded configuration. The at least one expanded configuration may comprise a freely expanded configuration and an in-use expanded configuration. The absorbent device may be configured to transition to the in-use expanded configuration when deployed inside the vaginal canal.

The catheter may further comprise a proximal electrode positioned proximally of the distal electrode assembly. A collective surface area of exposed portions of the distal electrode assembly may be less than a surface area of an exposed portion of the proximal electrode. The proximal electrode may comprise a first ring electrode, and the ablation catheter may further comprise a second proximal electrode comprising a second ring electrode. The first ring electrode may comprise a first conductive material, and the second ring electrode may comprise a second, different conductive material. A ratio between an exposed surface of the first proximal ring electrode and an exposed surface area of the second proximal ring electrode may be about 1:8 to about 1:4.

The insert electrode may be press-fit within the outer electrode. The insert electrode may be bonded to the outer electrode by a conductive adhesive, solder joint, or weld. Electrical coupling between the insert electrode and the outer electrode may be provided by direct metal-to-metal contact at an interface between the insert electrode and the outer electrode. The insert electrode may be fixed relative to the elongate body. A proximal portion of the insert electrode may be dimensioned such that the insert electrode occupies a majority of a cross-sectional area of a distal portion of the fluid lumen. The fluid lumen may house a conductive trace coupled to a proximal surface of the insert electrode. The insert electrode may comprise a first conductive material and the outer electrode may comprise a second, different conductive material. Fluid flow through the fluid passages may be configured to reduce or prevent clot formation at an interface between the insert electrode and tissue.

In some variations, an ablation catheter may comprise a shaft, a ring electrode positioned on the shaft and comprising a first diameter, and a distal electrode extending beyond a distal end surface of the catheter and comprising a second diameter, wherein a ratio of the first diameter to the second diameter may be about 0.15 to about 0.75. The shaft may comprise a third diameter, and the second diameter may be about ½ to about ¼ of the third diameter. The distal electrode may extend beyond the distal end surface of the catheter by up to 50% of the third diameter. The distal electrode may be configured for fluid flow therethrough. The distal electrode may comprise a rounded tip. The shaft may comprise a non-conductive polymer.

A high voltage conductor may extend through the fluid lumen and be electrically coupled to the distal electrode assembly, wherein the high voltage conductor may be electrically isolated from fluid within the fluid lumen. The high voltage conductor may be disposed substantially centrally within the fluid lumen. The high voltage conductor may extend through the entire length of the fluid lumen. The high voltage conductor may be configured to deliver pulsed-field energy to the distal electrode. A minimum radial distance between an outer surface of the high voltage conductor and an inner surface of the fluid lumen may be about 0.5 mm to about 4 mm. In some variations configured for smaller catheter profiles, a minimum radial distance between an outer surface of the high voltage cable and an inner surface of the fluid lumen may be about 0.1 mm to about 0.5 mm.

The high voltage conductor may be surrounded by at least one insulation layer. The at least one insulation layer may comprise one or more of polyimide, PEEK, PTFE, or ETFE. The fluid lumen may comprise a circular, oval, polygonal, or irregular cross-sectional shape. A ratio between a maximum transverse dimension of the high voltage conductor and a maximum transverse dimension of the fluid lumen may be about 1:8 to about 1:4. The high voltage conductor may comprise copper, stainless steel, platinum-iridium, or nickel-titanium. The high voltage conductor may be configured to deliver a pulse having an amplitude between about 2 kV and about 20 kV. The high voltage conductor may terminate at a proximal surface of or within the electrode. The high voltage conductor may terminate within a channel in the electrode.

The fluid lumen may deliver fluid to one or more outlets of the distal electrode assembly while the high voltage conductor remains fully insulated from the fluid. The high voltage conductor and the fluid lumen may extend through an articulating segment of the catheter configured to deflect. The high voltage cable may be spaced apart from an inner surface of the fluid lumen such that fluid delivered to a distal end of the elongate body flows around the high voltage cable. The high voltage cable may be positioned within the fluid lumen such that fluid delivered to the distal end of the elongate body circumferentially surrounds the high voltage cable as it flows along a length of the high voltage cable. The high voltage cable may comprise a high voltage conductor and one or more insulation layers around the high voltage conductor, and the high voltage cable may be floating within the fluid lumen.

An isolation manifold may be configured to direct the high voltage conductor into the fluid lumen. The catheter may further comprise a handle coupled to the elongate body and housing the isolation manifold therein, wherein the isolation manifold may be further configured to merge a fluid conduit within the handle and the voltage conductor to direct the voltage conductor into the fluid lumen. The isolation manifold may comprise a first inlet port coupled to the high voltage conductor, a second inlet portion coupled to the fluid conduit, and an outlet portion coupled with the fluid lumen of the elongate body. The isolation manifold may be positioned within the handle. The fluid conduit may be at least partially positioned within the handle.

The fluid lumen may be configured to guide fluid from the fluid conduit to a distal end of the catheter such that the fluid circumferentially surrounds the high voltage conductor. The high voltage conductor may be insulated along its entire length within the fluid lumen. The high voltage conductor may be surrounded by at least one insulation layer configured to maintain electrical isolation between the high voltage conductor and the fluid guided through the fluid lumen. The first and second inlet ports may converge to form the outlet port with a single shared lumen for the high voltage cable and the fluid. The manifold may comprise a Y-shaped, T-shaped, or curved junction between the first inlet port, the second inlet port, and the outlet port. The high voltage cable may be suspended within the fluid lumen. The fluid may comprise saline. The fluid conduit may be fluidly coupled to the fluid lumen via the isolation manifold.

Systems for delivering electrical energy to biological tissue may comprise a catheter having any of the features described herein and a deployment device configured to at least partially enclose the catheter and to maintain the ablation catheter in a compressed configuration. The deployment device may comprise a plunger configured to eject the catheter therefrom.

### BRIEF DESCRIPTION OF THE DRAWINGS

FIG. 1 depicts a perspective view of an exemplary ablation catheter including a handle, shaft, and a tip body.

FIG. 2A depicts a cross-sectional view of an isolation manifold and fiber optic connector positioned within a catheter handle. FIG. 2B depicts a perspective view of an isolation manifold for a catheter.

FIG. 3 depicts a side view of a catheter shaft illustrating proximal, middle, adjustable, distal end, articulating segment, and tip portions.

FIGS. 4A-4D depict cross-sectional views of various portions of a catheter shaft. FIG. 4A illustrates a proximal portion including pull wires, conductors, and an elongated lumen. FIG. 4B illustrates an adjustable portion including low-friction liners around pull wires. FIG. 4C illustrates a distal shaft portion including markers and a hypotube including a polymer layer thereon. FIG. 4D illustrates an articulating segment region showing conductor routing through a fluid lumen.

FIGS. 5A-5B depict a catheter distal portion. FIG. 5A illustrates a perspective view including cut-pattern features and proximal electrodes. FIG. 5B illustrates a longitudinal cross-section showing internal routing of an optical fiber.

FIGS. 6A-6B depict views of a tip body of the catheter. FIG. 6A illustrates a cross-sectional side view showing a proximal coupling interface and lumen. FIG. 6B illustrates a front cross-sectional view showing an outer perimeter and central lumen.

FIG. 7 depicts a back-side perspective view of the tip body with proximal coupling features.

FIG. 8 depicts a perspective view of a tip of the catheter showing a fluid lumen and conductors extending proximally.

FIG. 9 depicts a side view of a tip body showing a transition angle between the longitudinal axis and an insert electrode apex.

FIG. 10 depicts a front view of a tip body showing a triangular insert electrode defining three fluid passages with an outer electrode.

FIGS. 11A-11C depict variations of a triangular insert electrode. FIG. 11A illustrates a transverse cross-section with rounded vertices and concave sidewalls. FIG. 11B illustrates a side view showing proximal and distal portions and an apex. FIG. 11C illustrates a perspective view including a recess for conductor attachment.

FIG. 12 depicts a partial cutaway view of a tip body assembly with an insert electrode positioned within an outer electrode.

FIGS. 13A-13B depict a perspective and side, cross-sectional view respectively, of a catheter tip having a distally curved outer electrode.

FIGS. 14A-14B depict a perspective and side, cross-sectional view respectively, of a catheter tip having a triangular insert electrode seated in a shaped recess.

FIGS. 15A-15B depict a cylindrical or post-type insert electrode. FIG. 15A illustrates the electrode within the catheter tip. FIG. 15B shows a perspective view of the cylindrical or post-type electrode alone.

FIGS. 16A-16B depict a ribbed insert electrode. FIG. 16A shows the electrode within the catheter tip. FIG. 16B shows a perspective view of the ribbed insert electrode alone.

FIGS. 17A-17B depict a dome-shaped insert electrode with micro-ports. FIG. 17A illustrates the dome-shaped insert at the catheter tip, whereas FIG. 17B illustrates the dome-shaped insert alone.

FIGS. 18A-18B depict a pyramidal insert electrode. FIG. 18A illustrates a pyramidal insert electrode at the catheter tip, whereas FIG. 18B illustrates the pyramidal insert electrode alone.

FIG. 19 depicts a catheter tip having a perforated conductive plate electrode.

FIG. 20 depicts a catheter tip having an elongate insert electrode including micro-holes or diffusion apertures.

FIG. 21 depicts a schematic of an integrated tubular conductor forming at least part of a fluid lumen structure.

FIG. 22 depicts a cross-sectional view of an integrated tubular conductor embedded within a watertight polymer.

FIG. 23 depicts an alternative integrated conductor configuration having a fenestrated structure.

FIG. 24 depicts front, side and cross-sectional views of a tip body of a catheter showing electrode diameter relationships.

FIG. 25 depicts electric field distribution showing improved radial symmetry with optimized diameter relationships.

FIG. 26 depicts a flow diagram of a method for operating a catheter with controlled laminar flow irrigation.

FIG. 27 depicts a table of Reynolds number calculations for irrigation flow through passages defined by a triangular insert electrode.

### DETAILED DESCRIPTION

The disclosure herein is directed to catheter systems, components, and methods configured to deliver electrical energy to biological tissue (e.g., soft tissue). Ablation catheters are widely used across a range of therapeutic procedures, including, for example, cardiac ablation procedures targeting the pulmonary veins, ventricular tissue, or atrial tissue. In these and other contexts, delivering electrical energy through a precisely controlled field adjacent to the tip of the catheter may support targeted tissue modification while limiting unintended injury to nearby structures. The devices, systems, and methods described herein may be used in therapeutic procedures involving electrical energy delivery, including pulsed-field energy and radiofrequency energy, as well as procedures involving local electrical sensing or monitoring. The structural arrangements described below may be applied to a range of clinical targets and are not limited to a particular waveform, pulse shape, or energy modality.

Some procedures, including pulsed-field ablation (PFA) procedures, rely on delivery of high-voltage, short-duration electrical pulses that generate electric fields capable of inducing reversible or irreversible electroporation. These techniques may achieve tissue modification with reduced thermal effects compared to radiofrequency energy. However, because the tip of the catheter is exposed to substantial electrical and mechanical loads during use, many systems incorporate irrigation pathways to convey fluid to the tip. This fluid may cool the tissue interface, manage heat produced during energy delivery, and/or reduce coagulum formation. Providing adequate irrigation while maintaining a compact catheter geometry presents engineering challenges, particularly when multiple conductors, lumens, or insulating barriers must coexist within the same shaft.

Conventional catheter designs frequently route conductors and irrigation fluid through separate internal structures. For example, a conductor may extend through one lumen to energize a distal electrode, while a separate lumen provides irrigation fluid through outlets at or near the catheter tip. These arrangements can increase catheter shaft diameter, increase material requirements, and introduce structural interfaces that complicate manufacturing. The structural variations described herein may address these challenges by integrating electrical conductors and fluid pathways within a shared or adjacent lumen architecture. In some variations, a conductor may be routed through a fluid lumen and electrically isolated from the irrigating fluid by one or more insulating (e.g., dielectric) layers. Such integrated routing may permit the conductor to extend continuously from a proximal connector to a distal electrode while preserving a compact cross-sectional profile and reducing the number of internal partitions.

In some variations, a handle-integrated isolation manifold may be provided to merge a fluid conduit and at least one electrical conductor into a common fluid lumen that extends distally through the elongate body of the shaft of the catheter. The isolation manifold may maintain controlled spacing between the conductor and surrounding fluid conduit, may support fluid-tight transitions between proximal and distal fluid pathways, and may preserve conductor insulation under high-voltage operating conditions. Providing a defined isolation region at the handle may simplify manufacturing, reduce the risk of conductor damage at transition points, and support high-voltage delivery without additional reinforcing structures within the shaft.

The tip of the catheter may include a distal electrode assembly configured to deliver electrical energy and fluid at or adjacent to the tip. In some variations, the distal electrode assembly may comprise an insert electrode positioned within, or at least partially within, an outer electrode. The relative geometry of these components may define one or more fluid passages, may influence the radial or axial distribution of the electric field produced during operation, and/or may support controlled protrusion of the insert electrode with respect to the outer electrode. Configurations in which the insert electrode and outer electrode share a common central axis and are separated by a substantially uniform insulating portion may support more radially symmetric electric field distributions by reducing asymmetries that may otherwise arise in bipolar geometries with axially spaced or laterally offset electrode pairs. More uniform spatial field patterns may reduce catheter-orientation-dependent variability in field penetration depth, lesion contour, and/or activation threshold. Configurations in which the insert electrode has a smaller surface area than the outer electrode may concentrate electric field lines near the insert electrode and may promote a more radially uniform electric field distribution during bipolar energy delivery. By contrast with conventional bipolar arrangements that rely on axially spaced or laterally offset electrodes, coaxial arrangements in which the electrodes share a common central axis and maintain a substantially uniform radial separation may reduce orientation-dependent asymmetries in field penetration depth and lesion formation.

The integrated structural variations described herein—including the routing of conductors within a fluid lumen, the use of a handle—integrated isolation manifold, and the coaxial or nested geometry of the distal electrode assembly-may collectively address limitations associated with conventional catheter designs. These limitations may include asymmetric or orientation-dependent electric field distribution, increased shaft diameter due to separate fluid and conduction pathways, and increased thermal variability during energy delivery. The disclosed structures may therefore support reduced shaft diameters, simplified manufacturing, more predictable fluid dynamics at the catheter tip, and more uniform electric field characteristics across a range of catheter orientations.

The devices, systems, and methods described herein may be used in cardiac and non-cardiac therapeutic procedures, including electroporation-based therapies (reversible or irreversible), radiofrequency (RF)-based therapies, or other electrode-based energy modalities. While specific examples herein may reference pulsed-field energy delivery, the structural arrangements disclosed are not limited to a particular energy modality or waveform, and may be applied to a wide range of devices requiring integrated electrical and fluid delivery at a compact distal assembly.

As used in this specification and the appended claims, the term “distal” refers to a direction toward a work site, and the term “proximal” refers to a direction away from a work site. Spatially relative terms such as “beneath,” “below,” “upper,” “above,” “proximal,” “distal,” and the like may be used to describe relationships between elements as shown in the figures, but are intended to encompass variations in orientation or pose of the catheter during use. Geometric terms such as “parallel,” “perpendicular,” “round,” or “square” are not intended to require absolute mathematical precision, unless the surrounding context indicates otherwise. The singular forms “a,” “an,” and “the” include the plural forms unless the context clearly dictates otherwise, and the terms “comprises,” “includes,” “has,” and the like specify the presence of the stated features without precluding the presence or addition of other features.

Additionally, it should be appreciated that the ranges disclosed herein may be exemplary and include all ranges and subranges therein.

### I. Ablation System

In general, the system may comprise an ablation or monitoring/sensing device (e.g., ablation or mapping catheter) having a handle, a shaft, and one or more electrodes coupled to the shaft. The handle may be grasped and manipulated by an operator and may provide an interface for one or more connections to the remainder of the device, such as one or more electrical, fluidic, optical, and/or computational connections. The handle may additionally house mechanisms that actuate, control, or otherwise influence features at the distal end of the shaft, such as steering or deflection actuators and/or components configured to control the flow of irrigation fluid.

The shaft may extend distally from the handle and may include one or more lumens, channels, and/or structural regions configured to house electrical conductors, optical fibers, deflection components, or combinations thereof. In some variations, the shaft may comprise a plurality of contiguous shaft segments having different mechanical properties (e.g., stiffness, flexibility, torque transmission) and/or may be formed from different materials (e.g., different polymeric materials, different metallic materials, combinations thereof) to affect flexibility along the shaft. In some variations, the shaft may comprise a non-conductive polymer. In some variations, the shaft may include one or more pull wires or other actuation members extending through the shaft to facilitate steering or deflection of the catheter tip.

Furthermore, the catheter may comprise one or more electrodes. In some variations, the one or more electrodes may be positioned on a tip body that may be coupled to a distal end of the catheter shaft. In other variations, the electrodes may be positioned on a tip of the catheter via a distal end of the shaft itself. The one or more electrodes may be configured for ablation and/or sensing. The distal end of the shaft, or in variations comprising electrodes on the catheter tip, may include one or more fluid outlets (e.g., for irrigation fluid), one or more sensing elements (e.g., optical or force-sensing elements), or combinations thereof. In some variations, the catheter tip or distal end of the shaft may include a distal electrode assembly that receives electrical energy via one or more conductive elements (e.g., conductive trace, high voltage cable comprising a conductor) extending through the shaft and may include an irrigation structure in fluid communication with an irrigation lumen extending proximally through the shaft. Additionally, or alternatively, the catheter tip or distal portion of the shaft may comprise one or more electrodes positioned proximally along the catheter tip or distal portion relative to a distalmost surface of the catheter tip or distal portion (or relative to the distal electrode assembly).

In some variations, the catheter may comprise a distal tip defined by an insert electrode. The distal tip may be surrounded by a proximally positioned outer electrode, with each electrode positioned around or within the distal region of the shaft. The insert electrode may comprise a diameter that is smaller than that of the relative to the outer electrode. The outer electrode may have a diameter that is equal to, greater than, or less than an outer diameter of the shaft (or other body supporting the insert and outer electrodes) depending on the desired field-shaping configuration. In some variations, the outer electrode may be slightly larger than the shaft, while in others it may be inset or flush. The electrodes may be formed from biocompatible conductive metals, and in some variations, the insert electrode may incorporate apertures to permit irrigation fluid communication.

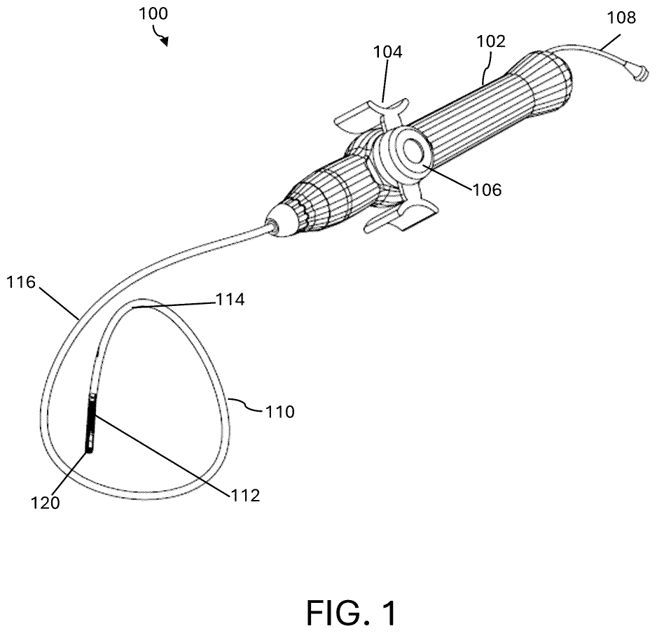

FIG. 1 depicts an exemplary ablation or sensing device 100. The device 100 may include a handle 102, a shaft or elongate body 110 coupled to and extending distally from the handle, and a tip 120 coupled to and extending distally from the shaft 110. The shaft 110 and the tip 120 may be coupled via an articulating segment 112. The handle 102 may be operable by a user to move the shaft 110 and/or the tip 120. For example, as shown, an adjustable portion 114 of the shaft 110 may be deflected/articulated. This deflection/articulation may be achieved using one or more adjustment mechanisms, e.g., adjustment mechanisms 104, 106 of the handle 102.

Detailed exemplary variations of the ablation system and aspects thereof are elaborated below.

### A. Handle

The devices described herein may comprise a handle configured to serve as an interface for user-controlled actuation, steering, or tensioning of one or more components that extend through the catheter shaft and/or tip. In some variations, the handle may be configured to be grasped and manipulated by an operator and may include adjustment mechanisms or actuators to control deflection of the catheter shaft and/or tip. For example, these adjustment mechanisms or actuators may include one or more sliders, knobs, levers, rotational elements, or other controls operable to adjust tension, compression, or displacement of steering elements routed distally through the shaft. Such controls may allow selective bending or deflection of the shaft and/or tip.

The handle may also include one or more coupling interfaces for external systems. For example, the handle may be releasably couplable to a fluid source, e.g., via an irrigation conduit or pump line, and may include an inlet port configured to direct fluid into one or more shaft lumens. In some variations, the handle may be couplable to one or more electrical sources, including a pulsed-field generator, mapping console, or other system providing therapeutic or diagnostic energy. In further variations, e.g., when the systems include fiber-optic sensing or shape-sensing elements, the handle may be couplable to optical energy sources or detectors.

For example, referring again to FIG. 1, the handle 102 may comprise a plurality of adjustment mechanisms or actuators, such as a deflection mechanism 104 and a tension mechanism 106. The deflection mechanism 104 may be operable (e.g., moveable) by a user to move the adjustable portion 114 of the shaft 110, and in turn the catheter tip 120. To do so, the deflection mechanism 104 may be operably coupled to one or more pull wires (not shown) routed through the handle 102 (e.g., a distal portion thereof), the shaft 110, and through at least a portion of the catheter tip 120. The wire(s) may be coupled to a portion of the tip 120 (e.g., to a pull ring thereof) such that adjusting tension of the wire(s) adjusts an orientation, e.g., deflection, of the tip 120. The tension mechanism 106 may be operable to further adjust and/or lock the tension of the wire(s) to thus maintain an orientation of the catheter tip 120.

The handle may further house one or more circuits, controllers, switching elements, or passive or active components configured to coordinate electrical routing, impedance monitoring, and/or energy-delivery safety functions. In some variations, the handle may incorporate structures for strain relief, cable management, or fluid routing. Such components may be integrated within internal cavities of the handle or distributed between proximal and distal handle regions. For example, referring again to FIG. 1, the handle 102 may comprise a port for an external fluid conduit 108 to couple to the handle 102.

In some variations, the handle may further comprise a proximal connector configured to receive a removable or reusable fiber-optic cable. This connector may permit an optical cable to couple to the catheter without requiring the optical cable to be permanently integrated into the device. Providing a releasable optical interface may allow expensive optical assemblies to be reused, may simplify cable management during operation, and may reduce waste associated with single-use catheters.

The connector may be positioned on a proximal region of the handle and may be offset from user-operated controls to keep the optical cable out of the operator's working area. The connector may comprise an optical coupler, keyed interface, or locking geometry that aligns and secures the proximal end of an optical fiber extending from the distal end of the catheter. In some variations, the optical fiber may extend continuously from the distal tip region to the connector, with the handle including internal routing or strain-relief structures that accommodate a small allowable excess length while maintaining bend-radius requirements and optical performance.

The connector may maintain precise axial and rotational alignment to preserve optical transmission characteristics and may be configured for releasable engagement so that the external fiber-optic cable may be detached and reused across procedures. Internal handle features may manage any excess fiber length, reducing the likelihood of sharp bends or stress concentrations.

Additionally, or alternatively, the catheter handle may comprise an isolation manifold may be configured to merge, route, or reorganize pathways in a manner that preserves controlled spacing, insulation integrity, and stable alignment of components extending distally through the handle, shaft, and tip body. The isolation manifold may be positioned within a proximal region of the handle, or at an interface between the handle and the shaft.

The isolation manifold may include a manifold body having a plurality of inlets and an outlet. Each inlet may be configured to receive a respective component, such as a fluid conduit or a conductive element (e.g., a conductive trace, a high-voltage cable), which may be positioned through the inlet and secured by, for example, press-fit engagement, adhesive bonding, interference fit, threaded engagement, snap-fit features, overmolded capture, or other coupling mechanisms. The outlet may be configured to interface with one or more lumens extending through at least a portion of the handle, shaft, and tip. That is, the outlet may be generally aligned with a lumen extending distally through the handle, shaft, and tip, and may be fluidly coupled thereto.

In some variations, the manifold may include first and second inlet ports that converge to form an outlet port. The first inlet port may be oriented at an angle relative to the second inlet port, such as at an angle of about 15 degrees to about 90 degrees, about 30 degrees to about 75 degrees, or about 45 degrees to about 60 degrees.

In some variations, the isolation manifold may be configured to merge fluid-delivery and electrical pathways into a common distal lumen while maintaining electrical isolation between them. The manifold may receive, at a proximal side, one or more fluid conduits and one or more conductive elements (e.g., a conductive trace (also referred to herein as a “conductor”), a high-voltage cable comprising a conductor, etc.) arriving from independent lumens, channels, or connectors. For example, a proximal portion of the manifold may include a fluid inlet and a conductor inlet arranged to allow a fluid conduit and a high-voltage electrical cable to enter the manifold separately and converge toward a single downstream lumen terminating in the outlet port. The cable may extend from its inlet port to the outlet port without requiring wire reversal, sharp bends, or disruption of insulation integrity. This configuration may prevent corona discharge effects that could otherwise occur at tight radius bends in high-voltage conductors. The manifold may maintain the conductor in a substantially straight or gently curved path as it transitions from the first inlet port to the outlet port, thereby preserving the electrical insulation surrounding the conductor.

The electrical cable, which may, in some variations, be a high-voltage cable, may include one or more dielectric coverings, jacketing layers, and/or shielding layers. Thus, fluid entering the manifold (e.g., from the second inlet port) may pass distally around the insulated conductor and through the fluid lumen of the catheter shaft while maintaining electrical separation from the conductor. In some variations, the manifold may include one or more seals, dielectric barriers, spacing elements, or internal structural features configured to maintain isolation between fluid and electrical components. Additionally, or alternatively, the manifold may maintain coaxial or near-coaxial positioning of the conductor relative to the shaft's fluid lumen to promote symmetric fluid distribution around the conductor during irrigation. Further, providing a direct, continuous routing of the cable through the manifold may reduce the need for splices or abrupt directional changes and may help preserve insulation integrity, minimize partial-discharge risk, and prevent corona formation during high-voltage operation.

The inlets and outlet of the isolation manifold may each comprise a circular, oval, polygonal (e.g., triangular, square, pentagonal, hexagonal), crescent-shaped, partially rounded, or irregular cross-section. In some variations, the outlet may be tapered, necked-down, or funnel-shaped to align the insulated cable within the lumen. Maintaining radial spacing between the cable and lumen wall may support electrical isolation and reduce stress on the cable. In some variations, the isolation manifold may comprise a branched manifold body having a Y-shaped, T-shaped, or L-shaped configuration.

In some variations, the manifold may be monolithic. In other variations, it may include a plurality of discrete parts that interlock or couple together. In some variations, the manifold may include an internal transition region in which separate inlet lumens for a fluid conduit and a conductor merge into a single shared lumen. This transition may be tapered or stepped. In other variations, the manifold may include one or more internal guide structures, ribs, or flow-directing walls that position or isolate the conductor as it transitions into the shared lumen.

The manifold may be formed from polymeric materials, metallic materials, ceramic materials, composite materials, or any suitable material capable of supporting fluid routing, electrical isolation, or mechanical transition into the shaft. In some variations, the manifold may be flexible or semi-rigid. The manifold may be attached to the handle or shaft by adhesive bonding, mechanical fastening, welding, overmolding, friction fitting, or other assembly techniques. Strain-relief features or flexible transition regions may also be incorporated to protect the cable during handling or operation.

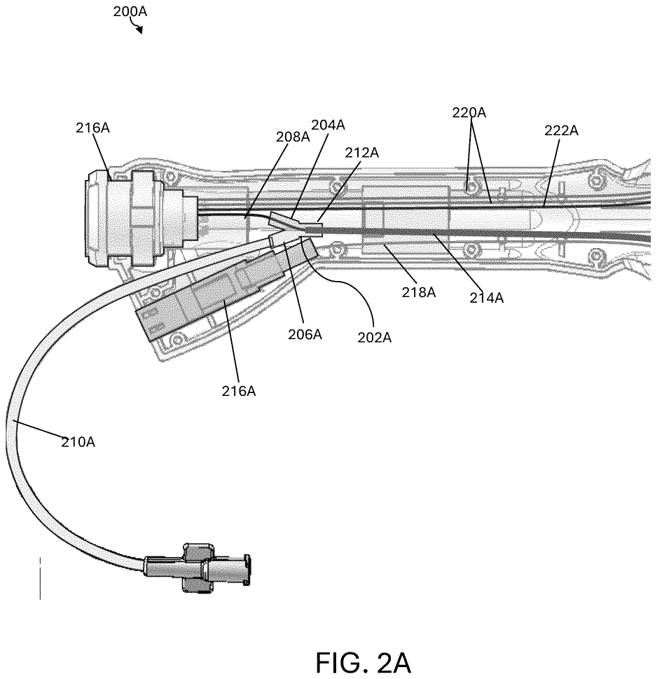

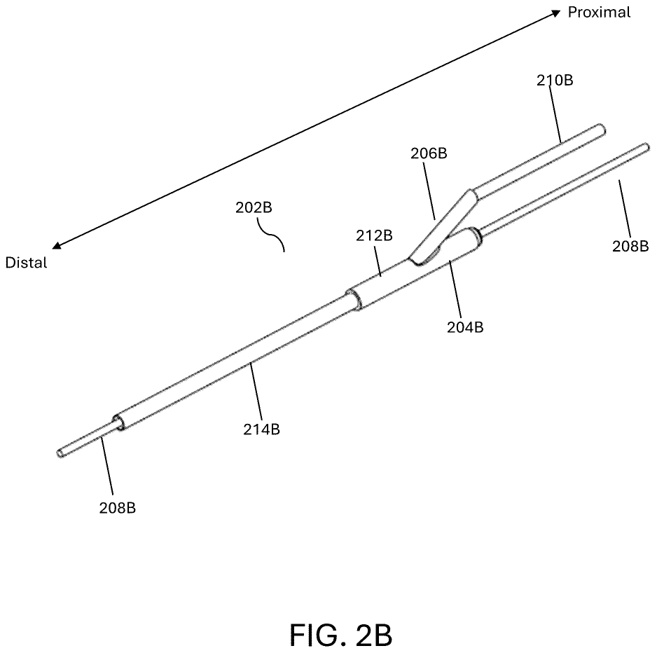

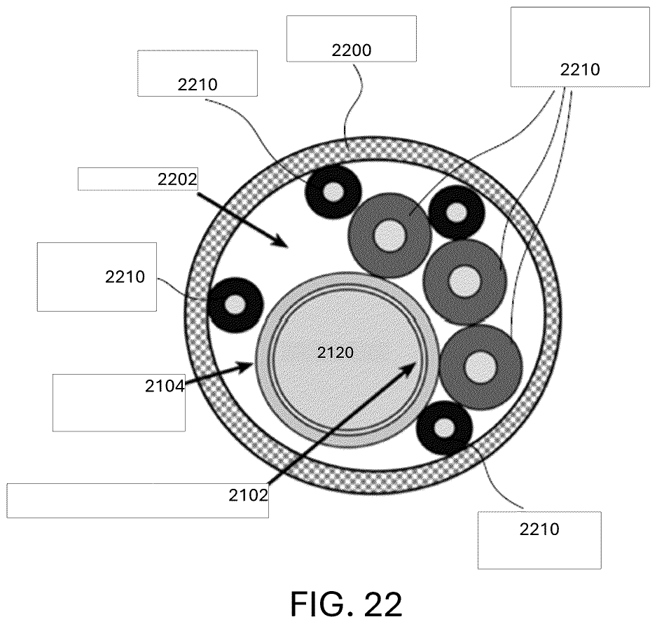

A schematic representation of an isolation manifold 202A within a catheter handle 200A is provided in FIG. 2A. The isolation manifold 202A may be positioned within any suitable portion of the handle 200A, such as, within a proximal region, central region, or distal region of the handle 200A. In some variations, it may be beneficial for the isolation manifold 202A to be positioned within a proximal region of the handle 200A as this may place it in closer proximity to electrical connector 216A. The manifold 202A may include a first inlet 204A comprising a lumen therethrough configured to receive or otherwise couple to an electrical cable 208A (e.g., comprising a conductor and insulation) and a second inlet 206A comprising a lumen therethrough configured to receive or otherwise couple to a fluid conduit 210A. The electrical cable 208A may be positioned within and extend through the first inlet 204A and continue distally into and through a lumen in the outlet 212A of the manifold and into the shaft's fluid lumen 214A. A proximal end of the electrical cable 208A may be electrically coupled with the electrical connector 216A configured to couple the electrical cable 208A to an external energy source, such as, for example, a generator. The fluid conduit 210A may enter the manifold through the second inlet 206A or may otherwise be coupled to the second inlet 206A such that fluid may pass distally through the outlet 212A (through the outlet lumen) into the same fluid lumen 214A as the electrical cable 208A, while remaining electrically isolated from the electrical cable 208A. This configuration is similar in principle to the integrated conductor design shown in FIG. 22, where electrical and fluid pathways are combined to reduce overall catheter complexity.

As shown, the manifold 202A may direct both the electrical cable 208A and fluid from the fluid conduit 210A into the shared outlet 212A and fluid lumen 214A coupled thereto without exposing the electrical cable 208A to sharp directional changes. The electrical cable 208A may thereby remain substantially straight or gently curved as it transitions into the shaft, reducing the likelihood of insulation damage or high-voltage discharge occurrences. The fluid may flow around the insulated cable 208A through the lumen 214A, with the manifold geometry maintaining separation and preventing fluid ingress into adjacent electrical components.

As further shown in FIG. 2A, the handle 200A may include a proximal optical connector 216 configured to receive a removable fiber-optic cable 218A. The connector 216A may optically couple the external cable (not shown) to an optical fiber 218A routed distally through handle 200A. The connector may maintain optical alignment, provide strain relief, and allow the optical fiber 218A to be engaged or disengaged without damaging the optical fiber 218A.

FIG. 2A additionally depicts conductors 220 that may electrically couple with one or more electrodes positioned along the catheter shaft (e.g., each to one electrode positioned proximal to a distal tip of the catheter). In some variations, the handle 210 may house conductors configured for delivery energy and/or sensing electrical signals. The conductor 218, for example, may be routed through the shaft and to a sensor (e.g., an EM sensor) at or within a distal tip body.

FIG. 2B depicts a perspective view of an isolation manifold 202B. Like the isolation manifold 202A, the isolation manifold 202B may comprise a first inlet 204B and a second inlet 206B that converge toward an outlet 212B. The first inlet 204B and second inlet 206B may be oriented at an angle relative to each other, which may be about 15 degrees to about 90 degrees, such as about 20 degrees to about 80 degrees, about 30 degrees to about 75 degrees, about 40 degrees to about 70 degrees, about 45 degrees to about 65 degrees, or about 50 degrees to about 60 degrees. As shown, an angle between centerline axes of the first inlet 204B and second inlet 206B may be about 45 degrees to about 60 degrees, though other angular configurations may be employed to accommodate different handle geometries and routing requirements.

The cross-sectional shapes of the first inlet 204B, second inlet 206B, and outlet 212B may vary depending on the components they are configured to receive and the desired flow characteristics. Although FIG. 2B shows generally circular cross-sections, the ports may alternatively comprise oval, elliptical, polygonal (e.g., triangular, square, pentagonal, hexagonal), crescent-shaped, partially rounded, spline-defined, or irregular cross-sections. In some variations, the outlet 212B may be tapered, necked-down, or funnel-shaped in a transition region configured to align or center components, such as an insulated electrical cable 208B, within a downstream lumen 214B. Different inlet ports may comprise different cross-sectional geometries; for example, the first inlet 204B configured to receive the electrical cable 208B may comprise a circular cross-section, while the second inlet 206B configured for fluid flow may comprise an oval or crescent-shaped cross-section to optimize distribution of fluid entering the manifold 202B.

Maximum transverse dimensions of the inlets and outlet may be selected based on the components routed through the manifold 202B. The first inlet 204B may have a maximum transverse dimension that is greater than, equal to, or less than a maximum transverse dimension of the second inlet 206B. In the variation shown, the first inlet 204B appears to have a greater maximum transverse dimension than the second inlet 206B, which may be advantageous when the first inlet 204B is configured to accommodate the electrical cable 208B and associated insulation. The outlet 212B may have a maximum transverse dimension that is about equal to the maximum transverse dimension of the first inlet 204B, as illustrated, to maintain consistent sizing of the electrical cable 208B as it transitions through the manifold 202B. In other variations, the outlet 212B may have a larger maximum transverse dimension than either inlet to accommodate both the electrical cable 208B and fluid flow around it, or may have a smaller maximum transverse dimension when constriction or flow regulation is desired.

As illustrated in FIG. 2B, the electrical cable 208B may enter through the first inlet 204B and may extend through the isolation manifold 202B to exit through the outlet 212B, maintaining a substantially straight or gently curved path to preserve insulation integrity and reduce stress concentration. A fluid conduit 210B may be received through the second inlet 206B, and fluid may flow through the manifold 202B to merge with the path of the electrical cable 208B within or proximate the outlet 212B. The manifold 202B may therefore define a transition region in which the separate pathways converge while preserving electrical isolation and spacing between the fluid and electrical components. Such a configuration may facilitate routing of both components into the common downstream lumen 214B extending distally through the catheter shaft while maintaining appropriate flow characteristics, insulation integrity, and positional stability.

### B. Shaft

The shaft may comprise an elongate body extending between the handle and the distal end of the catheter. The shaft may include one or more lumens providing electrical, mechanical, optical, and/or fluid pathways. In some variations, the shaft may be formed as a multilayer composite structure that includes one or more: polymeric bodies and/or layers (e.g., formed of polyether block amide (PEBA), polyethylene terephthalate (PET), polyamide, polyvinyl chloride (PVC), polyurethane (PU), silicone, combinations thereof or the like), braided or coiled reinforcement members, hypotube segments, lubricious liners or coatings, or insulative coatings.

The device may include one or more conductive elements (e.g., conductive traces, high-voltage cables) positioned within a lumen of the shaft or otherwise running therethrough. The one or more conductive elements may comprise copper, stainless steel, platinum-iridium, nitinol, composite conductors, or other biocompatible conductive materials. In some variations, one or more conductive elements may be individually insulated using polymer jackets, heat-shrink tubing, dielectric layers, or multilayer insulation. A conductive element may be disposed within the fluid lumen, or the fluid lumen may be routed centrally while conductive elements occupy peripheral positions. For example, a single fluid lumen may be used both for routing irrigation fluid and for routing a conductive element, e.g., an insulated high-voltage cable, thereby eliminating the need for separate dedicated conductive element and irrigation lumens. The conductive elements, e.g., high-voltage cables, electrically coupled with a distal electrode assembly may comprise one or more insulation layers sufficient to maintain electrical isolation at voltages between about 100 V and about 60 kV, between about 100 V and about 30 kV, between about 100 V and about 20 kV, between about 100 V and about 10 kV, between about 100 V and about 8 kV, between about 100 V and about 6 kV, between about 100 V and about 4 kV, between about 100 V and about 2 kV, between about 100 V and about 1 kV, between about 1 kV and about 60 kV, between about 1 kV and about 30 kV, between about 1 kV and about 20 kV, between about 1 kV and about 10 kV, between about 1 kV and about 8 kV, between about 1 kV and about 6 kV, between about 1 kV and about 4 kV, between about 1 kV and about 2 kV, between about 2 kV and about 20 kV, between about 4 kV and about 20 kV, between about 6 kV and about 20 kV, between about 8 kV and about 20 kV, between about 10 kV and about 20 kV, between about 10 kV and about 30 kV, between about 10 kV and about 40 kV, between about 10 kV and about 50 kV, between about 10 kV and about 60 kV, between about 12 kV and about 20 kV, between about 12 kV and about 25 kV, between about 12 kV and about 30 kV, between about 12kV and about 40 kV, between about 15kV and about 20 kV, between about 15 kV and about 25 kV, between about 15 kV and about 30 kV, between about 20 kV and about 40 kV, between about 20 kV and about 50 kV, between about 20 kV and about 60 kV, about 5 kV, about 10 kV, about 15 kV, and about 20 kV, including all ranges and sub-ranges therebetween.

In some variations, an insulated high-voltage cable routed within the fluid lumen may remain electrically isolated from fluid received in the lumen by one or more insulation layers that extend continuously along the length of the cable. Additionally, the high-voltage cable may extend continuously from a proximal electrical interface in the handle to a distal electrode assembly without intermediate splices, junctions, or electrical connections. This continuous routing may reduce potential failure points and maintain insulation integrity during energy delivery, such as, for example, high-voltage pulsed-field energy delivery.

In some variations, a conductive element may be positioned substantially centrally within the catheter shaft lumen and may retain substantially uniform radial spacing from the lumen walls. For example, a minimum radial distance between an outer surface of the conductor and inner surface of the fluid lumen may be about 0.25 mm to about 6 mm, such as about 0.5 mm to about 4 mm, about 0.75 mm to about 2 mm, or about 1 mm to about 2.5 mm. In some variations configured for smaller catheter profiles, a minimum radial distance between an outer surface of the high voltage cable and an inner surface of the fluid lumen may be about 0.1 mm to about 0.5 mm. In some variations, the minimum radial distance between an outer surface of the insulated conductor and an inner surface of the fluid lumen may be about 0.5 mm to about 5 mm, about 0.75 mm to about 4 mm, about 1 mm to about 3 mm, or about 1.5 mm to about 2 mm. A ratio between a maximum transverse dimension (e.g., diameter, major axis) of the insulated conductor and a maximum transverse dimension (e.g., diameter, major axis) of the fluid lumen may be about 1:12 to about 1:3, about 1:8 to about 1:4, or about 1:7 to about 1:5. This spacing and relative geometry may enhance circumferential fluid flow around the conductor and may maintain the conductor in a suspended or floating configuration within the lumen. In other variations, the conductor may be positioned within the fluid lumen without being aligned with the central longitudinal axis. In these variations, fluid may still flow circumferentially around the conductor.

In some variations, the conductive element may be maintained in a centered or near-centered position within the fluid lumen by internal supports, spacing features, or stabilized geometry of the lumen. Such features may help maintain radial spacing during bending or torsion of the shaft. That is, the geometry of the fluid lumen may be configured to maintain sufficient clearance around the conductor during shaft bending so that surrounding fluid flow and electrical isolation are preserved under flexed conditions.

In some variations, a conductive element may not merely pass through a fluid lumen but may itself define the fluid lumen structure. Specifically, the conductive element may comprise a metallic tubular element—such as a hypotube, braided tube, or coiled structure—that simultaneously provides electrical continuity and forms the conduit walls through which irrigation fluid flows. This integrated tubular conductor may eliminate the need for separate electrical and fluid-carrying structures by serving as both a high-voltage conductive element extending from the handle to the distal electrode assembly and as the structural boundary of a fluid pathway.

For example, in some variations, such a metallic tube may extend from a proximal region of the catheter toward a distal end of the shaft and may perform two functions: (i) defining a conduit for the flow of irrigation fluid, and (ii) providing an electrically conductive pathway between the handle and one or more distal electrodes. The metallic tube may comprise a polymer jacket that forms a watertight boundary capable of maintaining fluid integrity while electrically insulating the conductive metallic wall and provide electrical isolation while maintaining fluid containment, but the conductor itself may be the lumen-defining element rather than an element within a lumen. The integrated conductor may comprise one or more of stainless steel, nitinol, platinum-iridium, or other biocompatible conductive materials. The surrounding polymer jacket may comprise a medical-grade polymer with suitable dielectric properties. In some variations, the polymer jacket may have a conductivity less than about 0.1 micro-Siemens per centimeter. Additional polymer coatings or inner surface treatments may be applied to portions of the metallic component to reduce corrosion or limit interaction between the conductive structure and irrigation fluid. The conductive fluid lumen may therefore reduce the spatial requirements otherwise associated with separate insulated conductors and may simplify manufacturing by combining electrical and fluid transport within a single structural element. The metallic tube may comprise a solid metal tube, a tube incorporating discontinuities such as fenestrations or laser cuts, or a helical or braided wire structure arranged to define a lumen. The metallic tube may also provide rigidity and resistance to collapse, allowing the lumen to maintain patency throughout deflection and navigation.

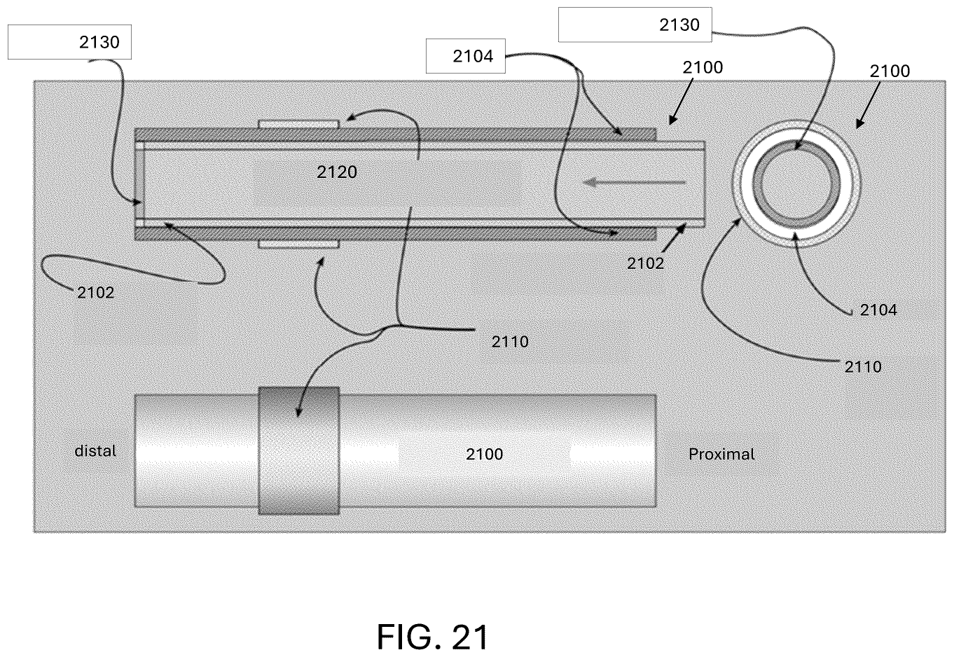

For example, FIGS. 21-23 illustrate catheter shafts 2100 having conductive element 2110 and fluid lumen 2120 configurations that minimize the space requirements within the shaft lumen. As shown in FIG. 21, the conductive element 2102 (e.g., a conductive trace) may comprise a metallic tubular component containing a fluid lumen 2120 that extends continuously from the handle through the shaft to a tip body electrode 2130. The metallic tubular component may facilitate the transfer of electrical energy from the handle of the catheter 2100 (not to the tip of the catheter). The metallic tubular element may be a solid metal tube, a tube that has discontinuities such as laser cutting or fenestrations, or metal braiding or coiling that takes the shape of a metal tube. The metallic tubular element may provide mechanical rigidity/strength to maintain the shape for the irrigation lumen 2120 and reduce the risk of kinking and/or collapse of the lumen. By providing such structural rigidity, the use of the metallic tubular component may allow the use of a more flexible polymer outer tube 2104 (e.g., dielectric). For example, the inclusion of the metallic tubular component may allow the use of watertight polymers and/or polymers that would otherwise be unsuitable due to undesirable mechanical qualities (examples too soft, too thin, too elastic to provide practical mechanical structure). The metallic tubular component may be encompassed by a polymer material (e.g., a dielectric polymer material) that may provide both electrical insulation and fluid containment. The polymer material may be any suitable medical grade polymer material and is configured to provide a fluid tight conduit to transport fluid from the handle to the tip of the catheter. The polymer material may also provide electrical insulation to ensure that the energy delivered via the metallic tubular component is delivered to the electrode without any arcing or escape paths. In some variations, the conductivity of the polymer material may be, for example, less than 0.1 micro-Siemens per centimeter. The polymer material may contact some or all of the outer surface of the metallic tubular component.

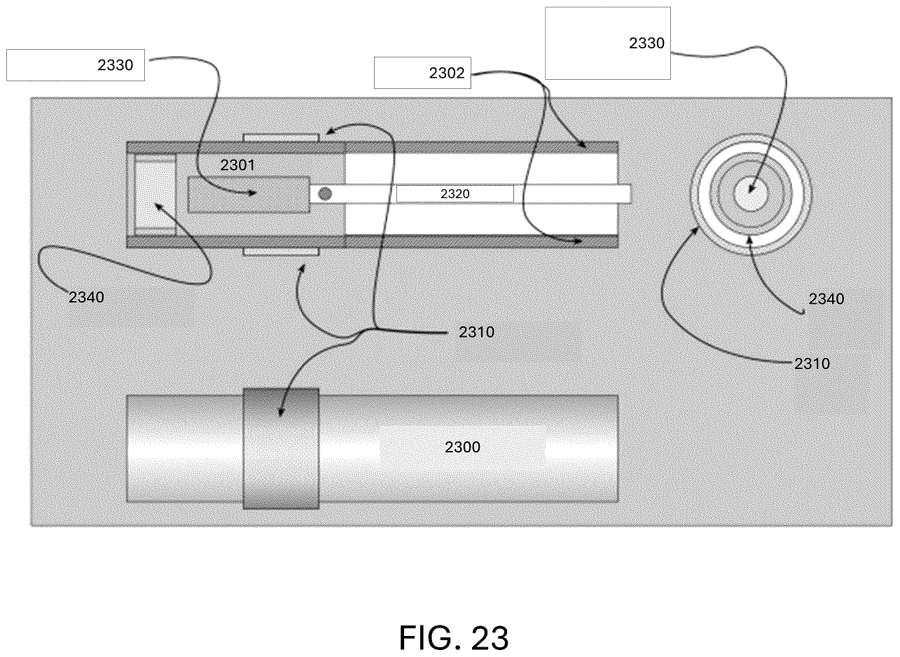

FIG. 22 shows a cross-sectional view of this configuration, illustrating how the metallic tubular component 2102 may be positioned within the watertight polymer tube 2104. Additional sensor wires and cables 2210 may be routed in the space 2202 between the outer catheter wall 2200 and the integrated conductor-fluid assembly. This arrangement may maximize an available cross-sectional area for fluid flow while maintaining electrical isolation. FIG. 23 depicts an alternative variation wherein the metallic conductor 2302 may include fenestrations or a braided structure while still maintaining tubular integrity.

Positioning a high-voltage cable within the fluid lumen 2320 may increase available isolation distance between the cable and other conductive elements in the shaft, including low-voltage traces, braided reinforcement members, or metallic structures. Maintaining this spacing 2301 may reduce the likelihood of dielectric breakdown, arcing, or partial discharge during pulsed-field energy delivery. Locating the high-voltage conductor 2330 centrally within a fluid lumen 2320 may also permit uniform surrounding fluid flow, which may help dissipate heat and maintain stable dielectric conditions during energy delivery.

The fluid lumen 2320 may deliver irrigation fluid distally to cool the distal electrode assembly 2330, 2340, stabilize electrical conditions at a tissue-electrode interface, or help distribute energy more uniformly during ablation. In some variations, the fluid lumen 2320 may also house one or more conductive elements, which may be fully insulated or partially exposed depending on configuration. Fluid may flow distally around any such conductors. The shaft may further house one or more pull wires extending toward the tip body.

One or more pull wires may extend longitudinally through dedicated lumens in the shaft and may be anchored to or engaged with a deflection mechanism in the handle. Manipulation of an actuator of the handle may apply tension to one or more pull wires to produce controlled bending or steering of an adjustable portion of the shaft.

The lumens or tubular components of the shaft may have circular cross-sections or may have non-circular forms such as oval, polygonal, crescent-shaped, or scalloped geometries. Non-circular lumen shapes may increase available cross-sectional area, accommodate asymmetrically positioned conductors, modify bending stiffness, increase torque response, or increase spatial separation between conductors and fluid-carrying structures. As an example, pull-wire lumens may include liners, reinforcement rings, or structural features configured to maintain patency and prevent lumen collapse during bending or steering of the shaft.

The shaft may have a length configured to access a target tissue of a patient while an operator is holding the handle. For example, the shaft may have a length of about 20 cm to about 180 cm, about 40 cm to about 160 cm, about 60 cm to about 140 cm, or about 80 cm to about 120 cm. A maximum transverse dimension of the shaft may be about 1 mm to about 5 mm, about 1.5 mm to about 4 mm, or about 2 mm to about 3.5 mm. Dimensions may vary depending on the number of lumens, electrode configuration, reinforcement structure, insulation thickness, or steering mechanisms.

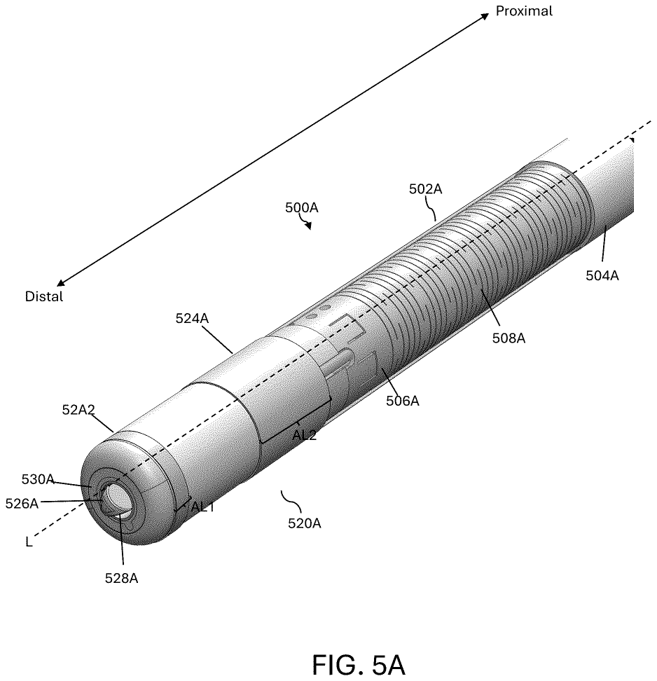

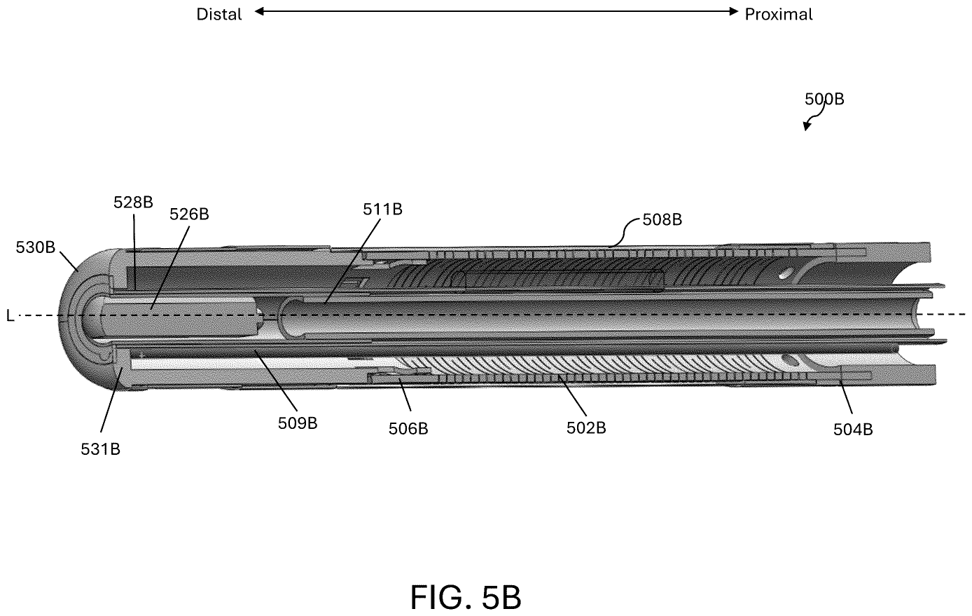

In some variations, the shaft may comprise multiple portions having different mechanical properties or internal arrangements. For example, the shaft may include a proximal portion coupled to the handle, a middle portion, an adjustable portion configured for deflection, and an articulating segment positioned distally of the adjustable portion and configured to couple to the catheter tip. The articulating segment may comprise proximal and distal ends and an elongate body extending therebetween. The articulating segment may incorporate flexible materials, reinforcement structures, or mechanical transitions that support pull-wire routing, fluid delivery, conductor positioning, and load transfer. For example, mechanical transitions between shaft portions may be formed by varying material durometers, reinforcement density, braid patterns, wall thickness, and/or cut patterns. These transitions may allow the shaft to change stiffness while maintaining lumen continuity. In some variations, the articulating segment may comprise a flexible hypotube. In other variations, the articulating segment may comprise a spring. The articulating segment is shown and described in more detail with reference to FIGS. 5A and 5B below.

The adjustable portion of the shaft may be configured to deflect under tension applied through one or more pull wires. In some variations, the adjustable portion may achieve bidirectional deflection of up to about 180 degrees in one or both directions and/or may provide multi-degree-of-freedom deflection, including compound curvature or off-axis bending. The adjustable portion may be formed from a hypotube (e.g., hypotube comprising a cut pattern configured to increase flexibility), a flexible polymeric section, a segmented reinforcement structure, and/or a composite architecture configured to maintain luminal patency during bending.

The articulating segment may be a flexible coupling element that mechanically links the distal end of the shaft to the proximal end of the tip body of the catheter. The coupling element may include a hypotube, a flexible polymeric structure, a coil-reinforced section, or another flexible connector. The coupling may be achieved by interference fit, adhesive bonding, welding, snap-fit features, crimping, press-fitting, or combinations thereof. The articulating segment may accommodate transitions in stiffness, maintain electrical isolation, and ensure continuity of fluid lumens, conductor pathways, and pull-wire routing between the shaft and the tip. In some variations, the articulating segment may not be needed and the tip may be directed coupled to the distal end of the shaft.

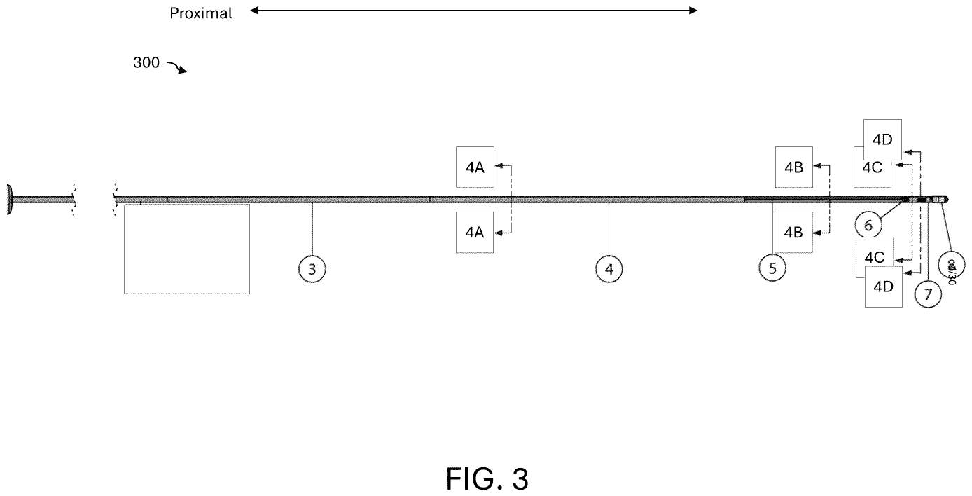

Exemplary portions of the catheter and catheter shafts described herein are illustrated in FIGS. 3 to 5B. In FIG. 3, a side-view of a catheter shaft 300 is shown that may include a proximal portion 3, a middle portion 4, an adjustable portion 5, a distal end portion 6, an articulating segment 7, and a tip body 8. The portions may differ in flexibility, material structure, or internal component arrangement.

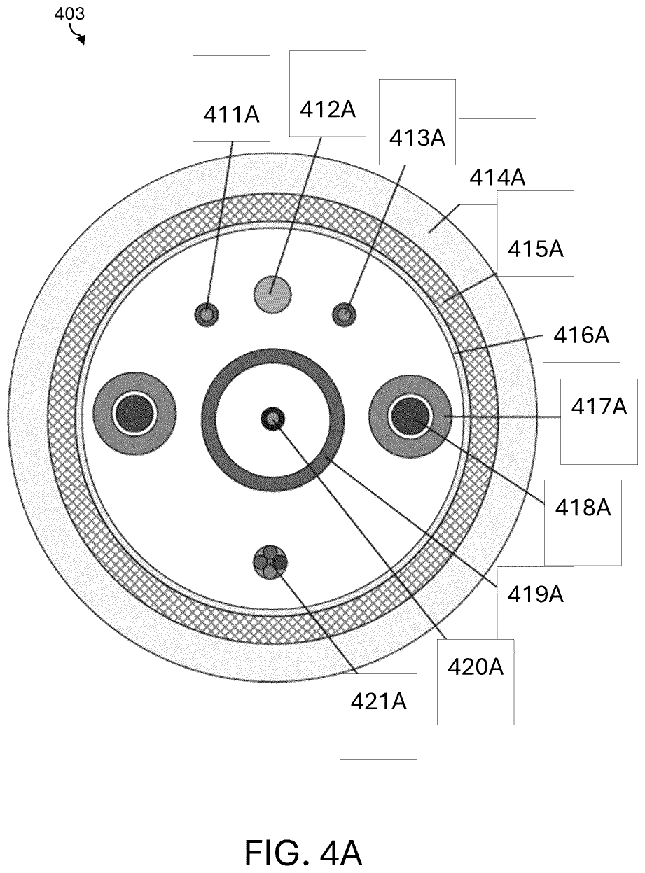

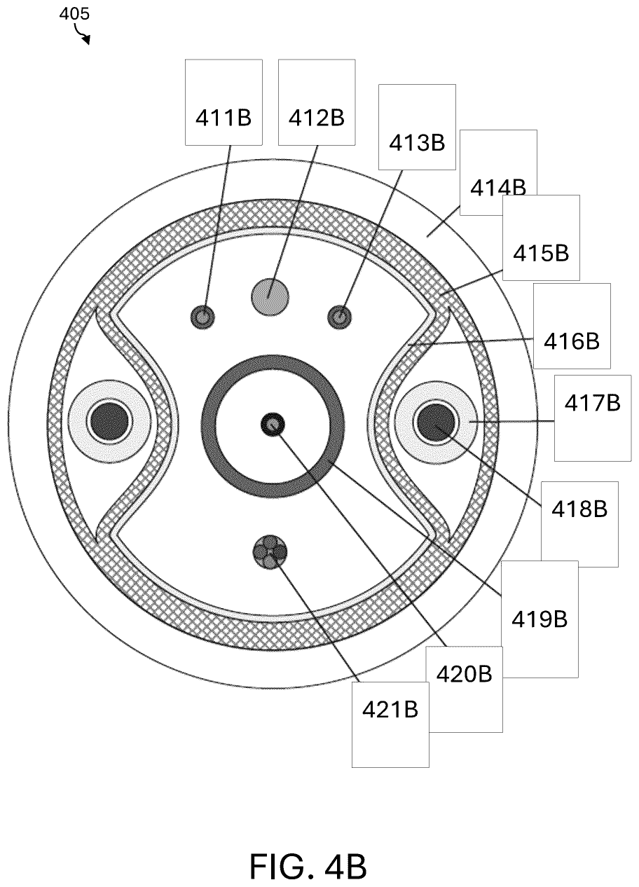

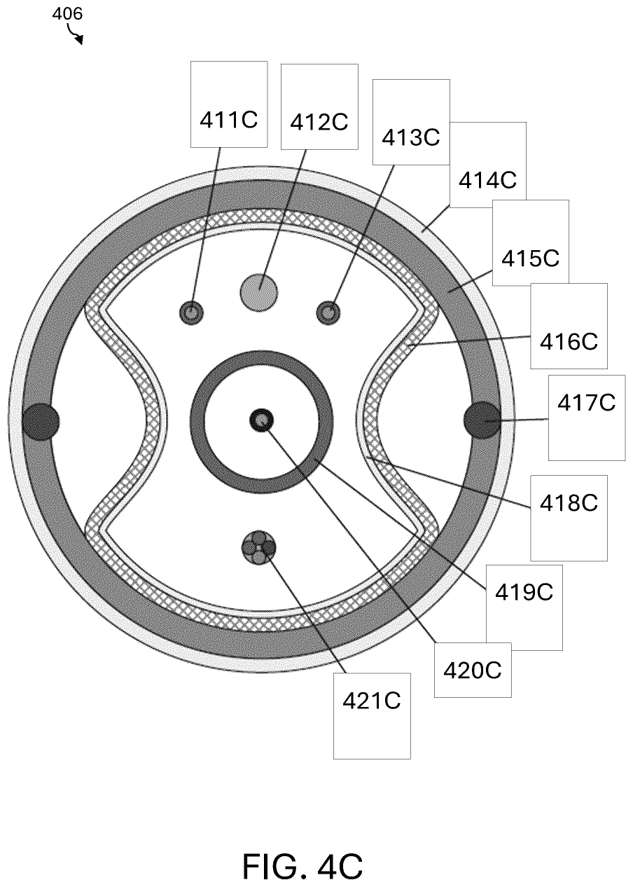

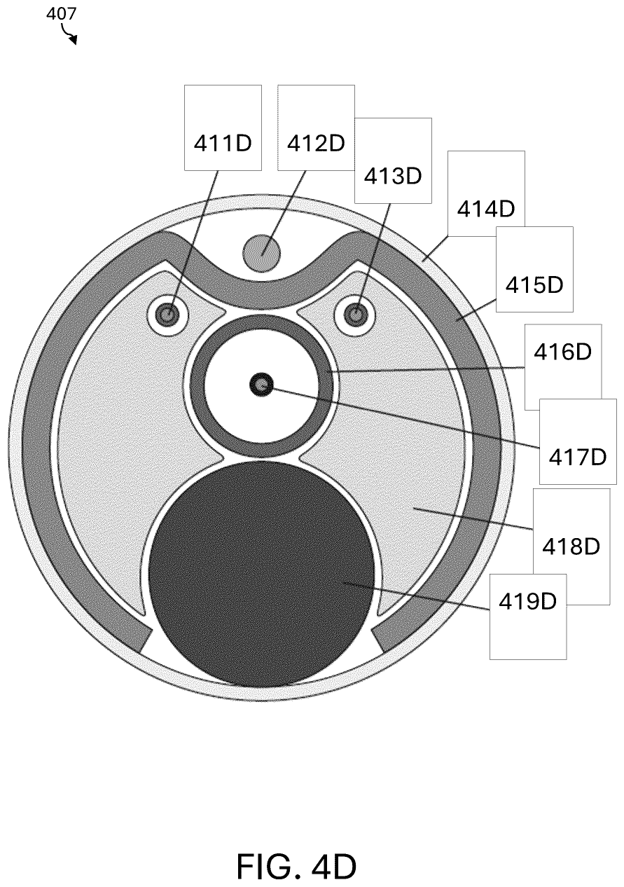

FIG. 4A depicts a cross-sectional view of an exemplary proximal portion 403 of the catheter shaft 300 along the 4A-4A line of FIG. 3. As shown there, the proximal portion of the shaft 300 may comprise the following components routed therethrough: a plurality of pull wires 418A (e.g., two, three, four, or more), a plurality of corresponding compression coils 417A around the respective pull wires 418A, high voltage conductors 411A, 413A, and 420A, each configured for energy delivery to one or more shaft electrodes (e.g., distal electrode assembly, proximal electrodes), one or more optical fibers 412A, and one or more magnetic sensor conductor cable or bundle of cables 421A. The proximal portion 403 may comprise a fluid lumen 419A therethrough, which may, as depicted in FIG. 4A, contain a high voltage conductor 420A positioned therein. In some variations, the proximal portion 403 may further comprise an outer jacket or extrusion 414A, a reinforcement braid 415A, and an inner shaft liner 416A.

FIG. 4B depicts a cross-sectional view of an exemplary adjustable portion 405 of the catheter shaft 300 along the 4B-4B line of FIG. 3. This portion may include a plurality of pull wires 418B each surrounded by a low friction liner 417C, high-voltage conductors 411B, 413B, 420B, one or more optical fibers 412B, an outer jacket 414B, a reinforcement braid 415B, an inner shaft liner 416B, a central fluid lumen 419B, and a magnetic sensor conductor cable or bundle of cables 421B. The geometry of the adjustable portion 405 may enable controlled deflection while maintaining separation between electrical, mechanical, and fluid pathways. The adjustable portion 405 may be more flexible than the proximal portion 403, which may be due at least in part to the low friction liners around the pull wires.

FIG. 4C depicts a cross-sectional view of an exemplary tip body 406 of the catheter along the 4C-4C line of FIG. 3. This region may include a visualization marker (e.g., radiopaque marker such as a tungsten band, printed marker) that delineates the end of the adjustable portion 405 when viewed using, for example, x-ray and/or fluoroscopy. The tip body 406 may comprise a plurality of pull wires 417C configured for connection to a pull ring positioned on the shaft and/or the articulating segment, high-voltage conductors 411C, 413C, 420C, one or more optical fibers 412C, a flexible hypotube 415C and an outer polymeric layer or cover 414C therearound, a reinforcement braid 416C, an inner shaft liner 418C, a central fluid lumen 419C, and a magnetic sensor conductor cable or bundle of cables 421C. The tip body 406 may be configured for mechanical and electrical coupling to an articulating segment or directly to the catheter shaft 300. The tip body 406 may be less flexible than the adjustable portion 405.

FIG. 4D depicts a cross-sectional view of an exemplary articulating segment 407 along the 4D-4D line of FIG. 3, which may be positioned between a distal end of the shaft 300 and the proximal end of a tip body 406 of the catheter. The articulating segment 407 may include a central fluid lumen 416D, high-voltage conductors 411D, 413D, 417D, one or more optical fibers 412D, cover or outer layer 414D over a flexible hypotube 415D, flexible potting material 418D, and a magnetic sensor 49D or sensor assembly. As depicted, the high-voltage conductor 417D may be positioned within the fluid lumen 416D. The articulating segment 407 provides mechanical, electrical, and fluidic continuity between the shaft 300 and the tip body 406 while accommodating angular flexibility and maintaining electrical isolation between components. The articulating segment 407 may be more flexible than the tip body 406, and about equally or less flexible than adjustable portion 405.

FIG. 5A depicts a perspective view of a distal portion of a catheter 500A. The catheter 500A may comprise an articulating segment 502A comprising an articulating segment body 508A extending between proximal and distal ends 504, 506A of the articulating segment 502A. The articulating segment 502A may join the distal end of the shaft to the proximal end of the tip body 520A while maintaining continuity of one or more lumens configured for fluid flow, conductor routing, or pull-wire passage. As shown, the articulating segment body 508A may include one or more cut patterns (e.g., laser-cut features), fenestrations, or slots configured to tune the flexibility, torsional response, and/or bending stiffness of the articulating segment 502A. These features may define longitudinal or circumferential segments that permit controlled deformation under tension applied through pull wires while helping maintain the patency and relative alignment of internal lumens.

The articulating segment 502A may be concentrically aligned with a central longitudinal axis L of the catheter and may interface with the tip body 520A through a snap-fit, press-fit, adhesive bond, weld, interference fit, or combinations thereof. The articulating segment 502A may also be dimensioned to transition between the mechanical characteristics of the more proximally reinforced shaft portion and the tip body 520A. For example, the articulating segment 502A may include regions of differing wall thicknesses, material stiffness, or reinforcement density to provide a gradual mechanical transition that reduces stress concentrations during deflection of the tip body 520A.

FIG. 5B depicts a cross-sectional view of the distal portion of a catheter 500B, illustrating the internal arrangement of components within the articulating segment 502B and the tip body 520B. The catheter 500B is shown in longitudinal cross-section, revealing the articulating segment body 508B extending between the proximal and distal ends 504B, 506B of the articulating segment 502A.

As described above, the articulating segment 502B may mechanically and fluidically couple a more proximal structure (e.g., a distal portion of the shaft, not shown) to the tip body 520B. The articulating segment 502B may incorporate structural features, such as the helical cut pattern shown in the articulating segment body 508B, to tune the flexibility and bending stiffness of the distal shaft region. The proximal end 504B and the distal end 506B of the articulating segment 502B may include coupling elements (e.g., recesses, steps, tapers, or flat interfaces) configured to achieve secure mechanical attachment, such as via interference fit, adhesive bonding, or welding, to the adjacent proximal shaft and tip body.

### C. Tip Body of the Catheter

The catheter may comprise a tip body configured to support one or more electrodes, conductive elements, sensing elements, fluid lumens, and/or mechanical components that interface with steering or deflection mechanisms (e.g., pull wires) within the shaft. For example, the tip body may define one or more lumens configured to receive components from the shaft elongate body, such as a lumen to receive a fluid lumen housing a high voltage conductor. As discussed above, the tip body may be coupled to a distal end of the shaft via an articulating segment and/or one or more coupling mechanisms (e.g., snap fit and/or adhesive). The tip body may be an elongate body that extends distally from the distal end of the shaft and/or articulating segment, and may have a length of about 2 mm to about 50 mm, such as about 3 mm to about 40 mm, about 4 mm to about 30 mm, about 5 mm to about 20 mm, or about 6 mm to about 10 mm. Moreover, a maximum transverse dimension of the tip body may be about 0.5 mm to about 8 mm, such as about 1 mm to about 6 mm, about 2 mm to about 4 mm, or about 2.5 mm to about 3.5 mm. In some variations, the maximum transverse dimension of the tip body may align with the maximum transverse dimension of the shaft (e.g., a distal portion thereof). For example, both of these dimensions may be about 2 mm to about 4 mm (e.g., about 3 mm).

The tip body may be formed from one or more polymeric, metallic, ceramic, or composite materials. In some variations, the cross-sectional shape of the tip body may be semi-circular, circular, oval, polygonal (e.g., triangular, square, hexagonal), partially tapered, or otherwise shaped suitably to accommodate internal lumens, sensors, and/or electrode structures.

The tip body may include one or more electrodes configured for one or more of: ablation, sensing, recording, and mapping. The electrodes may comprise conductive biocompatible metals such as platinum, platinum-iridium, gold, stainless steel, nitinol, or combinations thereof.

In some variations, one or more electrodes of the tip body may be configured to deliver high-voltage pulsed electric field energy. The voltage delivered to the tip during pulsed-field ablation, depending on the selected waveform and energy-delivery protocol, may be about a voltage of between about 100 V and about 60 kV, between about 100 V and about 30 kV, between about 100 V and about 20 kV, between about 100 V and about 10 kV, between about 100 V and about 8 kV, between about 100 V and about 6 kV, between about 100 V and about 4 kV, between about 100 V and about 2 kV, between about 100 V and about 1 kV, between about 1 kV and about 60 kV, between about 1 kV and about 30 kV, between about 1 kV and about 20 kV, between about 1 kV and about 10 kV, between about 1 kV and about 8 kV, between about 1 kV and about 6 kV, between about 1 kV and about 4 kV, between about 1 kV and about 2 kV, between about 2 kV and about 20 kV, between about 4 kV and about 20 kV, between about 6 kV and about 20 kV, between about 8 kV and about 20 kV, between about 10 kV and about 20 kV, between about 10 kV and about 30 kV, between about 10 kV and about 40 kV, between about 10 kV and about 50 kV, between about 10 kV and about 60 kV, between about 12 kV and about 20 kV, between about 12 kV and about 25 kV, between about 12 kV and about 30 kV, between about 12 kV and about 40 kV, between about 15kV and about 20 kV, between about 15 kV and about 25 k V, between about 15 kV and about 30 kV, between about 20 kV and about 40 kV, between about 20 kV and about 50 kV, between about 20 kV and about 60 kV, about 5 kV, about 10 kV, about 15 kV, and about 20 kV, including all ranges and sub-ranges therebetween.

In some variations, the tip body may comprise one or more proximal electrodes and a distal electrode assembly. The one or more proximal electrodes (e.g., two, three, four, five, six, seven, eight, nine, ten or more) may be disposed proximally of the distal electrode assembly, and may function as ablation electrodes, sensing electrodes, return electrodes, or combinations thereof. The distal electrode assembly may comprise one or more electrodes (e.g., first and second electrodes) arranged in coaxial, nested, overlapping, and/or interlocking configurations and may define one or more fluid-flow passages, field-shaping surfaces, and/or exposed conductive regions configured for energy delivery. The proximal electrodes and distal electrode assembly are described in detail below.

The tip body may additionally house internal components such as conductive elements, fluid lumens, pull wires, or other mechanical components that interact with one or more actuators located in the handle. For example, the tip may support distal portions of electrical cables routed through the handle, shaft, and tip to electrically couple to an electrode on the tip. In some variations, the tip body may comprise a pull ring (steering ring, anchor ring, collar), or other structure configured to receive the distal end of one or more pull wires.

Additionally, or alternatively, the tip body may house one or more sensors such as optical sensors configured for force sensing, electromagnetic (EM) sensors, impedance sensors, temperature sensors, or combinations thereof. For example, the tip body may house an EM sensor configured for navigation and localization. The EM sensor may comprise at least two degrees of freedom (DOF), such as at least three, four, five, or six DOF. In some variations, the force sensor comprises one or more optical fibers (one, two, three, four optical fibers). The one or more optical fibers may comprise on or more Fiber Bragg Gratings (FBGs) and may be positioned offset from a central longitudinal axis of the catheter.

For example, FIG. 5B depicts a cross-sectional view of the distal portion of a catheter 500B. This internal view illustrates the routing of a component, such as a force-sensing optical fiber 509B, which may be positioned offset from the central longitudinal axis L of the catheter 500B. The fiber 509B may extend distally through the articulating segment 502B and into the tip body 520B. The fiber 509B may extend to an attachment point that is at least partially embedded within a wall 531B of the tip body 520B, allowing it to sense strain/force near a distal-facing surface 530B of the catheter 500B. There, the component 509B may be adjacent to an outer electrode 528B and the insert electrode 526B, positioned at the distalmost end of the tip. Further, the fiber 509B may be routed within a dedicated, smaller lumen or channel (not shown) positioned within the articulating segment 502B and the tip wall 531B.

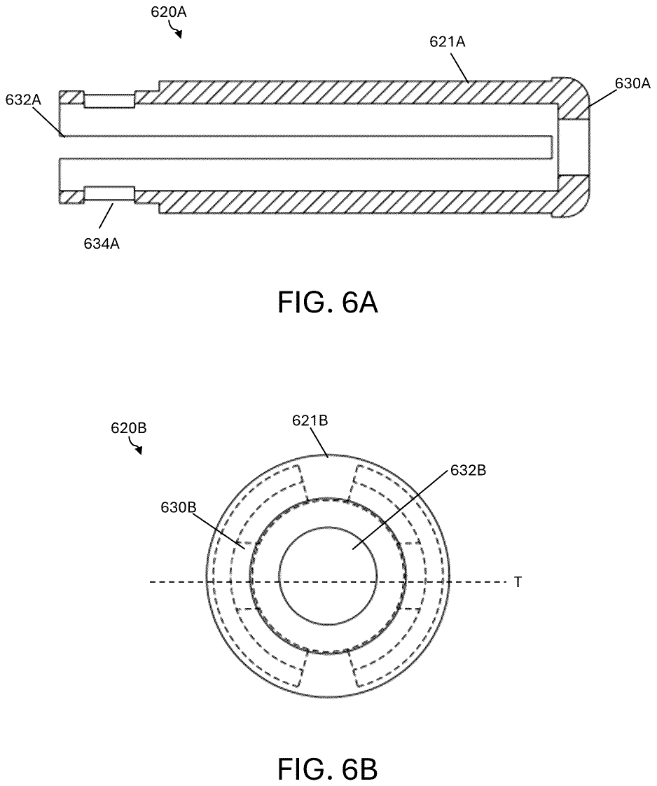

FIGS. 6A and 6B depict cross-sectional side and front views, respectively, of an exemplary tip 620A/620B. As shown in FIG. 6A, the tip 620A may comprise an elongate tip body 621A having a proximal portion 634A configured to couple to a articulating segment or distal end of a shaft (not shown). The coupling interface may comprise one or more complementary structures, such as an internal groove, recess, stepped geometry, interference surface, or adhesive-receiving region, that permits snap-fit engagement, press-fit engagement, adhesive bonding, or combinations thereof.

The tip body 621A may define at least one lumen 632A extending along a longitudinal axis of the tip body. In some variations, the lumen 632A may be a fluid lumen configured to route fluid toward the distal electrode assembly. Additionally or alternatively, the lumen 632A may be configured to provide a pathway for conductors and/or sensing elements. The tip body 620A may further include a distal face 630A, which may be planar, rounded, or tapered, and which may be shaped to support one or more distal electrodes. The wall thickness of the tip body 621A may vary along its length to accommodate internal reinforcement structures, conductor pathways, or desired stiffness transitions.

FIG. 6B depicts a cross-sectional front view of a tip 620B having a tip body 621B and a lumen 632B. As illustrated, the tip body 621B may define a generally circular outer perimeter, although other cross-sectional shapes (e.g., oval, polygonal, scalloped, or partially flattened geometries) may be used. The lumen 632B may be centrally positioned or laterally offset within the catheter depending on desired conductor spacing, fluid-flow distribution, or integration of additional lumens. The distal face 630B may define a perimeter surface configured to interface mechanically with a distal electrode assembly or an end-cap structure.

Transverse axis T is depicted with respect to the tip body 620B. The tip body 621A/B may have a maximum transverse dimension (e.g., diameter, major axis) of about 0.5 mm to about 8 mm, such as about 1 mm to about 6 mm, about 2 mm to about 4 mm, or about 2.5 mm to about 3.5 mm. A maximum transverse dimension of the lumen 632B (e.g., diameter, major axis) may range from about 0.2 mm to about 3 mm, such as about 0.4 mm to about 2 mm or about 0.8 mm to about 1.5 mm. Ratios between the maximum transverse dimension of the tip body and that of the lumen may range from about 2:1 to about 8:1, such as about 3:1 to about 6:1. These dimensional relationships may influence tip stiffness, thermal behavior, and routing of conductors or sensing components.

In some variations, a proximal portion of the insert electrode (not shown) may be positioned at least partially within lumen 632A/B and may be dimensioned such that the insert electrode occupies a majority of the cross-sectional area of the lumen. The remaining cross-sectional area may define an annular or segmental flow region configured to route fluid distally toward the fluid passages formed between the insert electrode and the outer electrode (described in detail herein).

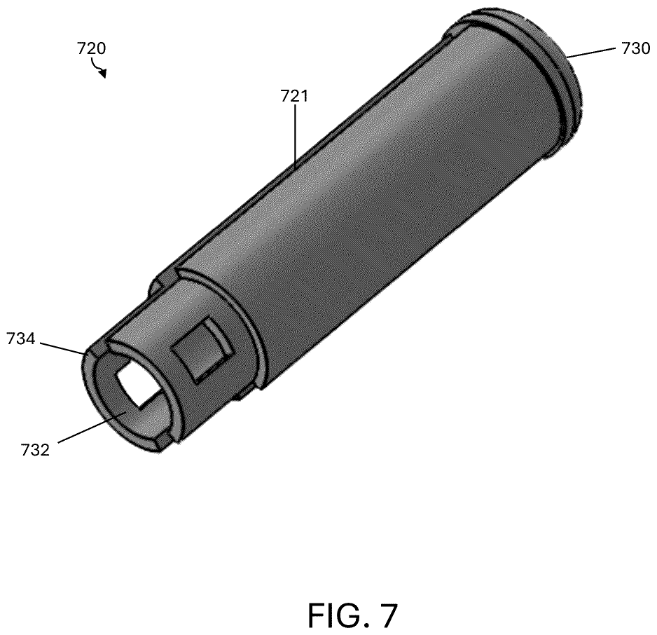

FIG. 7 depicts a back perspective view of a tip 720. The tip 720 may comprise a tip body 721 having a generally cylindrical outer surface and a proximal end 730 configured to couple with a shaft-side articulating segment. The proximal end 730 may define an internal cavity, recess, or step for receiving a corresponding articulating segment structure. These coupling features may permit snap-fit engagement, press-fit engagement, adhesive bonding, welding, thermal bonding, or combinations thereof.

An interior region of the tip 720 may define a lumen 732 sized to receive fluid, conductors, and/or a distal electrode insert. The lumen 732 may be circular, or may comprise any other suitable cross-sectional shape. In some variations, the lumen 732 may be a fluid lumen that routes fluid distally toward the distal electrode assembly and into one or more fluid passages formed between the distal electrode insert and the outer electrode. In some variations, the lumen 732 may house one or more insulated conductors.

As shown, the tip 720 may include coupling features at a distal portion 734 or a proximal portion 730 of the tip 720, which may be configured to mechanically interface with the articulating segment or a distal end of the shaft. Exemplary coupling features may include one or more cantilevered tabs, cutouts, windows, locking fingers, keyed geometries, or rotational alignment structures. Such features may maintain axial alignment between the tip body 721 and the articulating segment or shaft, preserve fluid-flow paths, and maintain desired spacing between conductors and insulating structures.

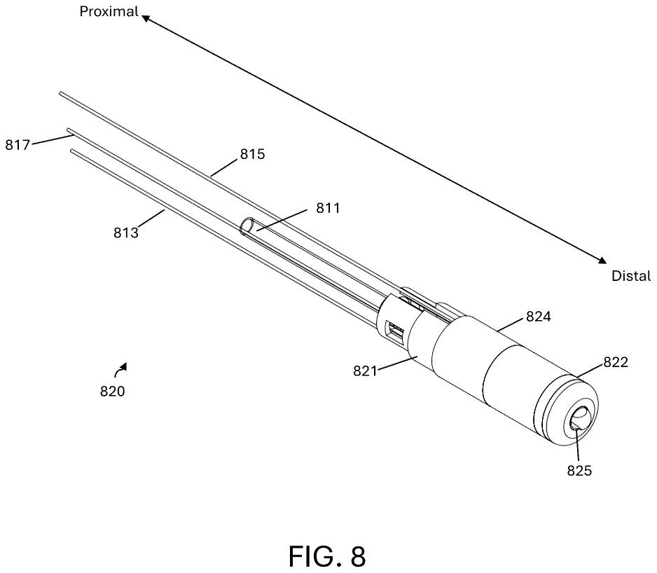

FIG. 8 is a perspective view of a catheter tip 820 with fluid lumen 811 and conductive elements 813, 815, 817 shown extending proximally therefrom. As shown, the conductive elements 813, 815, 817 may be routed in a generally parallel, longitudinally aligned arrangement along or within the shaft (not shown) and tip body 820. The conductive elements 813, 815, 817 may be spaced apart circumferentially or radially from one another to reduce electrical interference, minimize crosstalk, and maintain mechanical flexibility of the shaft and tip 820. In some variations, each conductive element 813, 815, 817 may correspond to a dedicated electrode of the tip 820 e.g., one conductive element routed to distal electrode assembly 825 and one conductive element routed to each of the first and second proximal electrodes 822, 824—such that the conductive elements may be individually activatable for sensing, mapping, or energy-delivery operations. The conductive elements may comprise insulated wires, cables, or coaxial structures, and may be supported or separated by internal ribs, lumens, partitions, and/or guide channels of the shaft, articulating segment, and/or tip. The fluid lumen 811 may be positioned centrally or off-axis relative to the conductors 813, 815, 817 and may provide a fluid flow path to the distal electrode assembly and/or to one or more irrigation outlets.

In some variations, the centrally positioned conductor 817 may be electrically coupled to the distal electrode assembly, whereas the additional conductors 813, 815 may be routed to the first and second proximal electrodes respectively. In some variations, one or more of the conductors (e.g., 813, 815) may be routed along or within sidewall-adjacent regions of the tip body 821 rather than within the fluid lumen 817, such as within dedicated trace channels, recessed guideways, or partitioned sidewall lumens formed within the tip body 821. Additionally, or alternatively, in some variations, the tip body 821 may comprise one or more auxiliary lumens or channels positioned along the sidewalls for routing conductive elements, or pull wires, thereby preserving the primary fluid lumen for irrigation flow while maintaining electrical separation and mechanical flexibility.

In some variations, one or more of the proximal electrodes 822, 824 may comprise a greater surface area than an exposed portion of the distal electrode assembly (i.e., the distal protruding tip and distal face of the outer electrode, as described below). In some variations, the first and second proximal electrodes 822, 824 when taken together may comprise a surface area greater than that of the distal electrode assembly.

### Proximal Electrodes

The one or more proximal electrodes may be arranged circumferentially, partially circumferentially, helically, segmentally, or in other geometric configurations on an outer surface of the tip body. In some variations, the one or more proximal electrodes may comprise a single ring electrode. In other variations, the one or more proximal electrodes may comprise a plurality of proximal electrodes arranged at uniform or non-uniform axial spacings along the tip body, and may have equal or unequal exposed surface areas. The number of proximal electrodes may range from one to ten or more, such as one, two, three, four, five, six, seven, eight, nine, or 10 electrodes. Each of the one or more proximal electrodes may function as an anode or a cathode depending on the energy-delivery protocol, may support mapping or sensing operations, or may perform combined sensing and energy-delivery functions.

Each proximal electrode may have any suitable geometry, such as a circumferential ring, partial ring, segmented ring, band, coil, mesh, or patterned electrode. The one or more proximal electrodes may be flush with the adjacent surface of the tip body, recessed into the tip body, protruding from the tip body, beveled, rounded, or shaped to influence local electric-field distribution. In some variations, one or more of the proximal electrodes may be flush, while one or more may be recessed or protruding.

An exposed surface area of each of the one or more proximal electrodes may be about 0.1 mm² to about 20 mm², including all values and sub-ranges therein. For example, the exposed surface area of each of the one or more proximal electrodes may be about 0.5 mm² to about 10 mm² , or about 1 mm² to about 5 mm². In some variations, the exposed surface area of each of the one or more proximal electrodes may be about 0.1 mm², about 0.2 mm², about 0.3 mm², about 0.4 mm², about 0.5 mm², about 0.6 mm², about 0.7 mm², about 0.8 mm², about 0.9 mm², about 1 mm², about 2 mm², about 3 mm², about 4 mm², about 5 mm², about 6 mm², about 7 mm², about 8 mm², about 9 mm², about 10 mm², about 11 mm², about 12 mm², about 13 mm², about 14 mm², about 15 mm², about 16 mm², about 17 mm², about 18 mm², about 19 mm², or about 20 mm². In general, a proximal electrode with a greater exposed surface area than the distal electrode assembly may contribute to a more uniform or more symmetric distribution of electric-field intensities around the distal region of the catheter. Axial spacing between a proximal electrode and the distal electrode assembly maybe about 0.1 mm to about 20 mm, such as about 0.5 mm to about 10 mm, or about 1 mm to about 6 mm. This spacing may define, in part, the degree of field concentration or symmetry during energy delivery.

In some variations, the tip body may comprise first and second proximal ring electrodes. The first proximal ring electrode may be positioned about 0.3 mm to about 1.0 mm from a distal end of the distal electrode assembly or the distal end of the tip body, or about 0.5 mm from the distal end of the distal electrode assembly or the distal end of the tip body. The second proximal ring electrode may be positioned about 1.5 mm to about 3.0 mm from the distal end of the distal electrode assembly or the distal end of the tip body, or about 2 mm from the distal end of the distal electrode assembly or the distal end of the tip body. The first proximal ring electrode may have a length of about 0.3 mm to about 1 mm, or about 0.5 mm, and a surface area of about 2 mm² to about 8 mm², or about 4 mm². The second proximal ring electrode may have a length of about 2 mm to about 5 mm, or about 3 mm, and a surface area of about 20 mm² to about 35 mm², or about 35 mm². A ratio between the surface areas of the first and second proximal ring electrodes may be about 1:8 to about 1:4, about 1:7 to about 1:5, or about 1:6.

Turning back to FIG. 5A, a geometry and relative spacing of first and second proximal electrodes 522, 524 is shown. The first proximal electrode 522 may be positioned immediately proximal to the distal electrode assembly 526, 528 and may have a preferred axial length AL1 of about 0.5 mm with a surface area of about 4.4 mm². The second proximal electrode 524 may be positioned proximally of the first proximal electrode 522 by about 2 mm and may have a preferred axial length AL2 of about 3 mm with a surface area of about 26.4 mm². As shown, the two proximal electrodes 522, 524 may be arranged coaxially about the longitudinal axis of the shaft and the tip body 520 and may be separated from the distal-facing surface 530 of the distal electrode assembly by axial offsets that correspond to the values described above. The relative axial lengths and surface-area ratio between the first and second proximal electrodes (e.g., about 1:6) may be selected to influence local electrical field distribution during pulsed electric field energy delivery and to maintain a substantially symmetric field profile around the tip body, regardless of approach angle.

### Distal Electrode Assembly

The distal electrode assembly may comprise two or more conductive structures arranged distally on the catheter, such as, for example, on the tip body or on the distal end of the shaft if a tip body is not employed. In some variations, the distal electrode assembly may comprise an insert electrode and an outer electrode arranged in coaxial, nested, and/or overlapping configurations. More generally, the distal electrode assembly may comprise any combination of conductive structures disposed at or near the distal end of the catheter (e.g., on a tip body) and configured to cooperate mechanically and electrically to facilitate pulsed electric field energy delivery, irrigation, sensing, mapping, or combinations thereof.

The distal electrode assembly may comprise one or more fluid-flow passages, electrode-surface transitions, or surface-area ratios configured to promote desired electric-field patterns and fluid dynamics. The relative geometry of the outer electrode and insert electrode may promote substantially laminar flow of fluid routed through the fluid lumen as the fluid enters and traverses the distal fluid-flow passages. In some variations, the fluid-flow passages may have a length and a diameter configured to establish laminar flow of fluid. For example, the fluid-flow passages may have a diameter of about 0.01 mm to about 0.035 mm, such as about 0.02 mm to about 0.03 mm, representing the gap or interstitial space between the insert electrode and the inner surface of the outer electrode. The length of the fluid-flow passages may correspond to the axial length along which fluid flows through these gaps, which may be about 1 mm to about 10 mm, such as about 2 mm to about 6 mm. This may result in a ratio of passage length to passage diameter that falls within a range configured to promote laminar exit characteristics, about 75:1 to about 250:1. Fluid-flow passages including a smooth inner surface (e.g., minimal or no surface irregularities) may also help establish laminar flow. The flow rate of fluid through these passages may be less than about 50 mL/min, such as less than 45 mL/min, less than 40 mL/min, less than 35 mL/min, less than 30 mL/min, less than 25 mL/min, less than 20 mL/min, less than 15 mL/min, less than 10 mL/min, about 10 to about 50 mL/min, about 15 to about 45 mL/min, about 20 to about 40 mL/min, about 25 to about 35 mL/min, about 25 to about 35 mL/min, about 20 mL/min, about 25 mL/min, about 25 mL/min, about 30 mL/min, about 35 mL/min, or about 40 mL/min (e.g., 30 mL/min). Maintaining laminar flow may help stabilize electrical conditions at the distal electrode assembly, promote uniform cooling of exposed conductive surfaces, reduce stagnation regions in which clot formation may otherwise occur, and support more consistent distribution of fluid around the distal electrode assembly. The distal electrode assembly may further be configured to reduce spatial asymmetry, mitigate field concentration at edges or boundaries, promote three-dimensional field uniformity, increase lesion controllability, and manage thermal conditions during energy delivery.

The distal electrode assembly may be configured with a smaller exposed surface area than one or both proximal electrodes (e.g., both combined) to promote predictable energy distribution and minimize unwanted current concentration at the distal tip. In some variations, the distal electrode assembly may comprise an exposed surface area collectively defined by a protruding distal tip and distal face of a surrounding outer electrode. The exposed surface area may be about 1 mm² to about 5 mm², such as about 2 mm² to about 4 mm², or about 2.5 mm² to about 3.5 mm².

Variations of the distal electrode assembly and aspects thereof are described below.

The outer electrode of the distal electrode assembly may generally be configured to surround at least a portion of the insert electrode. For example, the outer electrode may be cylindrical, tubular, or ring-shaped and may be positioned to encircle or otherwise surround at least a portion of the insert electrode. In some variations, the outer electrode may have a rectangular (e.g., square), triangular, polygonal, or irregular cross sectional shape. Additionally, or alternatively, the outer electrode may have a cross-sectional shape that varies along its length. In some variations, the outer electrode may extend proximally into the tip body and/or the elongate body of the shaft, may be partially recessed within the tip body and/or the elongate body of the shaft, may be flush with the distal surface of the tip body and/or elongate body of the shaft, or may protrude distally from the distal surface of the tip body and/or elongate body of the shaft to varying degrees. For example, in some variations, the outer electrode may extend distally from the distal surface of the tip body (extend distally of the catheter tip) up to about 50 percent of the diameter of the catheter shaft. The outer electrode may have a length of about 0.1 mm to about 10 mm, such as about 0.3 mm to about 5 mm, about 0.5 mm to about 3 mm, or about 0.7 mm to about 2 mm.

The outer electrode may comprise a circular, triangular, polygonal, oval, or irregular cross-sectional shape and may be dimensioned to surround at least a proximal portion of the insert electrode. In some variations, the outer electrode may have a maximum transverse dimension (e.g., outer diameter) of about 1 mm to about 5 mm, such as about 2 mm to about 4 mm, or about 2.5 mm to about 3.5 mm. For example, the outer electrode may have a maximum transverse dimension of about 3 mm. The inner transverse dimension (e.g., inner diameter) of the outer electrode may be configured to accommodate the insert electrode while maintaining the fluid passages therebetween in at least some locations. For example, the inner transverse dimension may be about 0.5 mm to about 4 mm, such as about 1 mm to about 3 mm, or about 1.5 mm to about 2.5 mm, depending on the dimensions of the insert electrode.

Further, the outer electrode may have a maximum transverse dimension that is substantially equal to, slightly larger than, or slightly smaller than the maximum transverse dimension of the adjacent tip body and/or elongate body of the shaft. In variations in which the outer electrode has a maximum transverse dimension (e.g., diameter) of about 3 mm and the central electrode has a maximum transverse dimensions (e.g., diameter) of about 1 mm, the ratio of the insert electrode maximum transverse dimension to the outer electrode maximum transverse dimension may be about 1:3. More generally, this ratio may be about 1:10 to about 1:2, such as about 1:8 to about 1:2.5, 1:6.5 to about 1:2.5, or about 1:5 to about 1:3.

In some variations, the outer electrode may at least partially surround a proximal portion of the insert electrode that is disposed within the tip body and/or elongate body of the shaft. In some variations, the insert and outer electrodes may be coaxially aligned around a central longitudinal axis of the shaft. The insert electrode and the outer electrode may be physically and electrically coupled. For example, the outer electrode may define an inner width or diameter or inner surface that is configured to engage with the vertices or outer perimeter of the insert electrode. Along the proximal portion of the insert electrode, each vertex may form a contact region (e.g., via interference fit, transition fit, press fit) with a corresponding portion of the inner surface of the outer electrode. The radial distance between the inner surface of the outer electrode and the outer surface of the insert electrode (at the vertices or contact points) may define the dimensions of the fluid passages, as described above.

In some variations, the outer electrode may terminate at a location that permits the insert electrode to extend beyond the distal end (e.g., distal-facing surface or distalmost edge) of the outer electrode. For example, in some variations, the insert electrode may extend about 0.05 mm to about 3 mm beyond the distal end of the outer electrode. The outer electrode may be positioned such that its distal end surface is substantially flush with, recessed from, or protruding beyond the distal end surface of tip body. The outer electrode may comprise the same or a different conductive material as the insert electrode. For example, the outer electrode may comprise platinum, platinum-iridium, gold, stainless steel, nitinol, or alloys thereof. The outer electrode may have a wall thickness of about 0.05 mm to about 1 mm, about 0.1 mm to about 0.5 mm, or about 0.15 mm to about 0.3 mm. The outer surface of the outer electrode may be smooth, textured, coated, or treated to enhance electrical conductivity, reduce thrombus formation, or improve tissue-electrode interface characteristics.

The insert electrode may generally comprise an elongated conductive member positioned within, adjacent to, or partially surrounded by an outer electrode. In some variations, a distal portion of the insert electrode may extend distally beyond a distal-facing surface of the tip body and beyond the distal-facing surface of the outer electrode. Put differently, the insert electrode may include a proximal portion that is housed at least partially within the tip body and/or elongate body of the shaft while a distal portion protrudes distally to form a tissue interface. The proximal portion of the insert electrode may be at least partially surrounded by a proximally-extending portion of the outer electrode. In some variations, the insert electrode may occupy a majority of a transverse cross-section of the outer electrode. The insert electrode may comprise any suitable conductive material, such as platinum, platinum-iridium, gold, stainless steel, nitinol, or alloys thereof, and may be solid, partially hollow, or perforated to support irrigation flow. The insert and outer electrodes may comprise a same or different material.

The shape of the insert electrode may define one or more fluid passages when positioned in spaced relation to the inner surface of the outer electrode. Put differently, the configuration of the insert electrode within the surrounding outer electrode may create fluid passages, lumens, channels, or gaps through which fluid routed through the fluid lumen of the shaft may flow distally and exit at or near the distal electrode assembly. The insert electrode may comprise a non-circular cross-section selected to balance electrical performance, irrigation characteristics, manufacturability, mechanical strength, and surface area. Suitable shapes may include polygonal profiles (e.g., triangular, square, pentagonal, hexagonal), curved or scalloped shapes, shapes having alternating concave and convex portions, shapes having three or more vertices or protrusions, or irregular shapes configured to define fluid passages of predetermined dimensions. In some variations, the insert electrode may comprise at least three vertices or protrusions that, when positioned relative to the inner surface of the outer electrode, create junctions or gaps forming fluid passages sized to permit fluid to flow between the vertices or protrusions and around the insert electrode.

In some variations, the insert electrode may comprise a triangular cross-section. Such a triangular configuration may include three vertices positioned at substantially equal or unequal angular intervals around a central longitudinal axis of the insert electrode. For example, vertices may be spaced at angular separations of about 60-180 degrees, about 80-150 degrees, or about 100-140 degrees (e.g., about 120 degrees). Sidewalls extending between vertices may be straight, faceted, curved (e.g. arcuate). For example, in some variations, the sidewalls may be concave. Concave sidewalls may increase an effective cross-sectional area of fluid passages and may have radii of curvature of about 0.01 mm to about 5 mm, about 0.05 mm to about 2 mm, or about 0.1 mm to about 1 mm. A triangular insert electrode may divide the fluid lumen into three discrete arcuate or crescent-shaped fluid passages, each positioned between a vertex of the insert electrode and an inner surface of the outer electrode. More generally, depending on the number and arrangement of vertices, the cross-section of each fluid passage may be semicircular, ovular, crescent-shaped, scallop-shaped, or may have another curved geometry. In some variations, each fluid passage may have a maximum transverse dimension of about 0.01 mm to about 1 mm, about 0.03 mm to about 0.5 mm, about 0.05 mm to about 0.3 mm, about 0.07 mm to about 0.2 mm, or about 0.1 mm to about 0.15 mm.

In some variations, fluid routed through the fluid lumen may be delivered directly to the proximal portion of the insert and/or outer electrodes and may flow distally through the fluid passages before exiting one or more outlets defined by the distal electrode assembly. In some variations, at least one of the outlets may be oriented to direct fluid flow radially outward from a central longitudinal axis of the elongate body of the shaft. Alternatively, in some variations, at least one of the outlets may be oriented to direct fluid flow radially inward from the central longitudinal axis. This may help ensure that the fluid is passed over the distal tip. The fluid may provide cooling, reduce thrombus formation, improve lesion uniformity, modulate electrical field distribution, or influence electrochemical conditions at the tissue-electrode interface. For example, the fluid may reduce or prevent clotting at the interface.

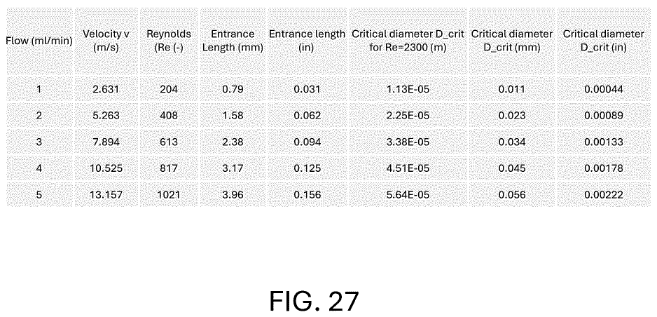

The distal electrode assembly may be configured to maintain laminar flow conditions (e.g., Reynolds numbers below about 2300) during fluid delivery. The noncircular cross-sectional geometry of the insert electrode, when positioned within the curved inner surface of the outer electrode, may form one or more discrete arcuate fluid passages having dimensions selected as described above. These passages may maintain Reynolds numbers between about 200 and about 1100 for flow rates of about 1 mL/min to about 5 mL/min, as demonstrated in FIG. 27. The plurality of passages may distribute fluid circumferentially around the electrode assembly while maintaining passage dimensions sufficiently small to ensure laminar conditions.

The relationship between the insert electrode and the inner surface of the outer electrode may interrupt what would otherwise be a continuous circular edge at the distal junction between the electrodes. Stated differently, the vertices of the insert electrode and the discrete fluid passages between the electrodes may break up a single continuous geometric edge into three or more separated regions. Each of the fluid passages may extend longitudinally along at least a portion of the insert electrode. This distributed geometry, particularly when combined with conductive irrigation fluid flowing through the passages, may promote more uniform electrical field distribution around the distal electrode assembly during ablation and may reduce localized field intensification that could otherwise occur at electrode edges or transitions. The resulting distributed edge effect may help mitigate focal tissue overheating, arcing, or unintended electrical concentration while maintaining efficient field penetration into target tissue.

As described above, the insert electrode may be positioned to extend distally beyond one or more adjacent structures of the catheter. For example, the insert electrode may protrude distally beyond a distal surface of the tip body and/or the elongate body of the shaft, may protrude distally beyond a distal surface of the outer electrode, and/or may extend distally through an opening defined by the outer electrode. An exposed or protruded length (i.e., the length of the insert beyond one or more adjacent catheter structures as described above) of the insert electrode may be about 0.01 mm to about 10 mm, about 0.03 mm to about 5 mm, about 0.05 mm to about 3 mm, about 0.1 mm to about 2 mm, about 0.3 mm to about 1.5 mm, about 0.4 mm to about 1 mm, about 0.5 mm to about 0.75 mm, or 0.5 to 1 mm. In some variations, the insert electrode may protrude by about 0.5 mm or at least about 0.5 mm. In some variations, the insert electrode may protrude about 0.15 mm to about 6 mm beyond the distal end surface of the outer electrode, such as about 0.2 mm to about 5 mm beyond, about 0.225 mm to about 4 mm beyond, about 0.25 mm to about 3 mm beyond, about 0.3 to about 2.5 mm beyond, about 0.4 to about 2 mm beyond, or about 0.5 to about 1 mm beyond the distal end surface of the outer electrode. The protruded portion of the insert electrode may include a distal apex formed by convergence of the vertices. The apex may be rounded, blunt, tapered, faceted, or otherwise shaped to facilitate tissue engagement, electrical field uniformity, or mechanical durability.

The insert electrode may define multiple geometric relationships with the catheter dimensions. An exposed or protruded length of the insert electrode may correspond to a portion of the insert electrode within a minimal geometric perimeter of other elements of electrical/fluid contacting elements of the catheter. A ratio between an exposed length of the insert electrode and a maximum transverse dimension of the tip body and/or the elongate body of the shaft may be about 1:12 to about 1:2, about 1:10 to about 1:3, about 1:8 to about 1:4, about 1:7 to about 1:5, or about 1:6.5 to about 1:5.5 (e.g., about 1:6). Further, a ratio between a maximum transverse dimension of the insert electrode and a maximum transverse dimension of the tip body and/or the elongate body of the shaft may be about 1:10 to about 9:10, about 1:5 to about 4:5, about 1:4 to about 3:4, about 1:3 to about 2:3, or about 2:5 to about 3:5. In some variations, the maximum transverse dimension of the tip body and/or the elongate body of the shaft may be about 1.5 to about 5 times the transverse dimension of the insert electrode. In some variations, the maximum transverse dimension of the insert electrode (e.g., a width or diameter thereof) may be less than about 90% of the maximum transverse dimension of the tip body and/or the elongate body of the shaft (e.g., a width or diameter thereof), such as less than about 85%, less than about 80%, less than about 75%, less than about 70%, less than about 65%, less than about 60%, less than about 55%, less than about 50%, less than about 45%, less than about 40%, less than about 35%, less than about 30%, or less than about 35% thereof.

In cases where the insert electrode has a diameter that is smaller than that of the outer electrode, the electric field distribution generated during bipolar operation may exhibit increased symmetry. The field density near the insert electrode may be elevated due to its reduced surface area, while the larger outer electrode may distribute the returning field lines across a broader region. This arrangement may reduce the dependence of field penetration depth on catheter orientation and may produce a more consistent therapeutic field contour. In some variations, these effects may emulate the more uniform field behavior observed between spherical electrodes of mismatched size in homogeneous conductive media.

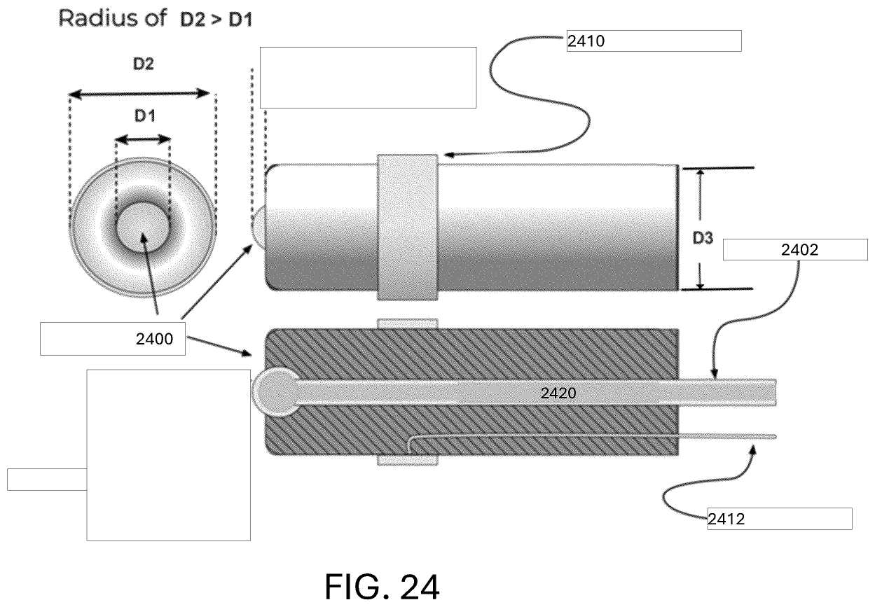

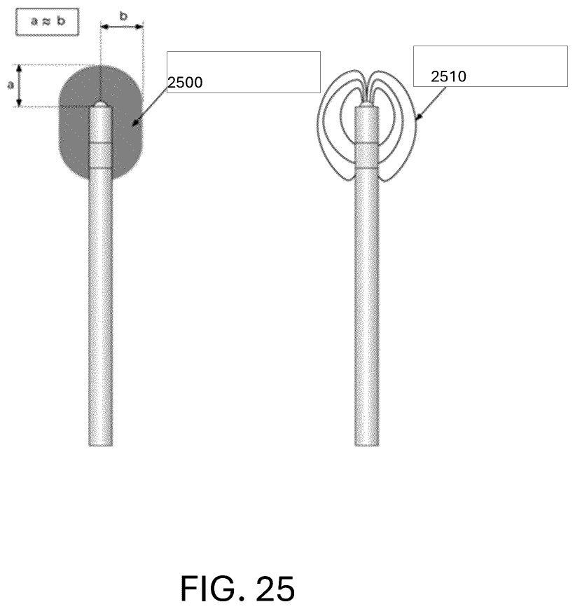

FIG. 24 depicts an electrode configuration where a tip electrode 2400 has a diameter D1 and a proximal ring electrode 2410 has a diameter D2, with the catheter shaft having diameter D3. Maintaining a ratio of D1:D2 of about 1:4 to about 1:2 may yield optimal field characteristics. The tip electrode 2400 may be flush with or protrude from the distal end by up to 50% of the shaft diameter D3. Thus, the deliberate size mismatch between electrodes 2400 and 2410 may promote a desired field amplification at the tip body. The electrode 2400 may be coupled to a first conductor 2402 (e.g., electrical trace), and the electrode 2410 may be coupled to a second conductor 2412 (e.g., electrical trace). The catheter shaft may optionally include an irrigation lumen (e.g., fluid lumen) 2420. FIG. 25 illustrates a resulting electric field distribution achieved with the diameter-optimized configuration depicted in FIG. 24. The approximated field contours 2500 demonstrate substantially improved radial symmetry compared to conventional bipolar designs where the tip electrode has equal or greater diameter than the ring electrode. For example, when considering a ring electrode positioned on the shaft comprising a first diameter and a distal electrode extending beyond a distal end surface of the catheter comprising a second diameter, a ratio of the first diameter to the second diameter may be about 0.15 to about 0.75. In particular, the field lines 2510 show uniform penetration depth (dimensions a and b being about equal) regardless of catheter orientation, addressing the asymmetry limitations of traditional bipolar catheters.

The geometry of the distal electrode assembly may further define transition angles with respect to the tip body and/or the elongate body of the shaft. For example, a transition angle measured between a longitudinal axis of the catheter and a transition axis extending from a point at an outer circumference of the elongate body to a point at a distal apex of the insert electrode may be less than about 60 degrees, less than about 45 degrees, less than about 30 degrees, or about 15 degrees to about 45 degrees (e.g., about 45 degrees). A shallow transition angle may promote smooth fluid flow transitions and reduce electrical field concentration at electrode-body interfaces.

In some variations, the ratio between the exposed length of the insert electrode and its maximum transverse dimension may also be selected to promote substantially symmetric electric-field distributions around the distal tip. For example, an exposed length that is on the order of about 0.25 to about 1.5 times a maximum transverse dimension of the insert electrode may allow field lines to emanate radially in a more uniform pattern, reducing preferential concentration at the distal apex or along any individual sidewall. By tuning this ratio together with the relative surface areas of the insert and outer electrodes, the distal electrode assembly may provide a substantially symmetric electric field about the longitudinal axis of the elongate body.

The insert electrode and outer electrode may be mechanically and electrically coupled to form a continuous or partially continuous conductive assembly. Coupling may be achieved through press-fitting, interference fitting, shrink-fitting, welding, brazing, soldering, conductive adhesive bonding, or diffusion bonding. In some variations, there may be direct metal-to-metal contact between the insert electrode and the outer electrode. Electrical coupling may occur along one or more interface or contact regions, including distal junctions where the insert electrode protrudes through the outer electrode, along overlapping or nested regions, or at discrete bonded portions of the electrodes. The electrical coupling may facilitate uniform current distribution across both electrode surfaces during ablation. For example, the distal electrode assembly may be electrically coupled to an electrical cable (e.g., a high voltage cable) via one or both of the outer and insert electrodes. For example, as described above, an insulated electrical cable may be routed through a fluid lumen of the catheter that is positioned proximal to or abuts a proximal end of the insert electrode. The cable may couple to the proximal end of the insert electrode (e.g., to an attachment point, such as a divot within the insert electrode body or to a proximal facing surface thereof) to transmit energy to the distal electrode assembly.

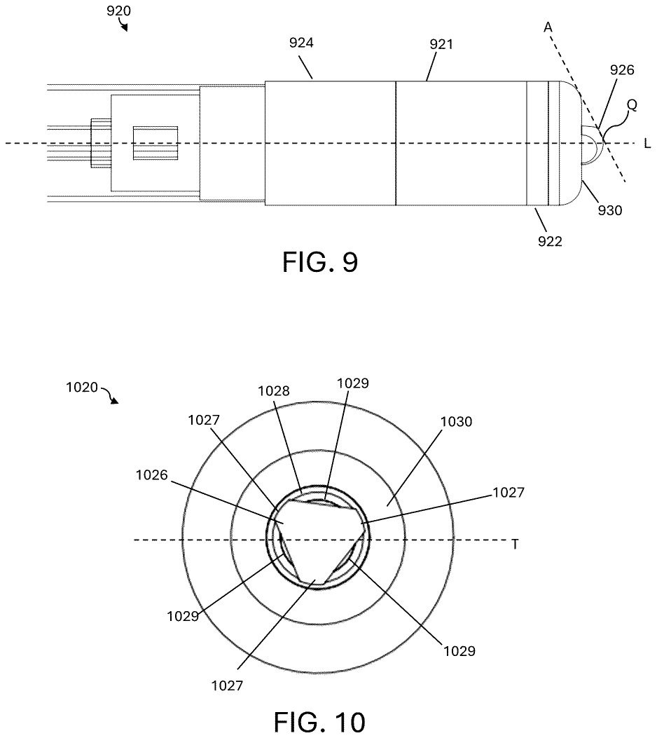

The following description referring to FIGS. 9-12 further discusses catheter tip bodies/distal electrode assemblies having insert electrodes with noncircular cross-sectional shapes that promote laminar or near laminar flow through the tip body.

FIG. 9 depicts a side view of a tip body 920 showing the longitudinal geometry of the tip body 921, the distal end region 922, and a proximal portion 924 of the shaft or articulating segment. The tip body 920 defines a longitudinal axis L extending through the device. A transition axis A extends from a point along an outer surface of the tip body 921 toward a distalmost region 926 of the insert electrode 930. An angle Q is defined between the transition axis A and the longitudinal axis L. In some variations, the angle Q may range from about 15 degrees to about 60 degrees, such as about 20 degrees to about 50 degrees, about 25 degrees to about 45 degrees, or about 30 degrees to about 40 degrees. This transition angle may facilitate smooth distal contouring, may reduce electrical field concentration at electrode-body junctions, and may promote atraumatic tissue interaction.

FIG. 9 further illustrates exemplary longitudinal placement of the distal electrode assembly relative to the tip body. The insert electrode 930 may protrude distally beyond an adjacent distal-facing surface of the outer electrode or tip body by about 0.1 mm to about 1.0 mm, such as about 0.2 mm to about 0.8 mm, or about 0.4 mm to about 0.6 mm. The tip body 921 may define a rounded or partially spherical contour at the distal region 922 with a radius of curvature selected to maintain smooth navigation and to influence distal field geometry.

FIG. 10 provides a front-facing view of a tip body 1020 showing an exemplary relationship between an insert electrode 1026 and the inner surface 1028 of an outer electrode 1030, and the multiple fluid-flow passages 1029 defined therebetween. As shown, the insert electrode 1026 may comprise a generally triangular or three-vertex transverse profile when viewed distally. Three vertices 1027 may be positioned around a central region of the insert electrode 1026 at substantially equal or unequal angular separations. The angular separation between adjacent vertices may range from about 80 degrees to about 150 degrees, such as about 100 degrees to about 140 degrees (e.g., about 120 degrees).

A transverse axis T is shown passing through the tip body, and the relative transverse dimensions of the components may be defined by reference to this axis. A maximum transverse dimension of the insert electrode 1026 may be about 0.5 mm to about 1.5 mm, such as about 0.8 mm to about 1.2 mm. A maximum transverse dimension of the outer electrode 1030 may be about 2 mm to about 4 mm, such as about 2.5 mm to about 3.5 mm. A maximum transverse dimension of the tip body may be about 2.5 mm to about 5 mm. Correspondingly, a ratio between the maximum transverse dimension of the insert electrode 1026 and the maximum transverse dimension of the outer electrode 1030 may range from about 1:10 to about 1:2, such as about 1:5 to about 1:3. In some variations, the ratio between the maximum transverse dimension of the insert electrode 1026 and the maximum transverse dimension of the outer electrode 1030 may be about 0.15 to about 0.75.

The interstitial regions 1029 formed between the vertices 1027 of the insert electrode 1026 and the opposing inner surface 1028 of the outer electrode 1030 define arcuate, crescent-shaped, or lobed fluid-flow passages. These passages may be bounded by smooth, continuous sidewalls of the insert electrode-such as curved surfaces having no protrusions, ribs, ridges, or abrupt transitions and by a smooth inner surface of the outer electrode. In some variations, the surfaces defining the passages may be free of sharp edges or steps to promote laminar flow, minimize internal flow disturbances, and reduce stagnation regions.

Each fluid-flow passage may have a maximum transverse dimension of about 0.01 mm to about 1.0 mm, such as about 0.02 mm to about 0.50 mm, about 0.03 mm to about 0.40 mm, or about 0.04 mm to about 0.30 mm. In additional variations, the maximum transverse dimension may be about 0.05 mm to about 0.30 mm, such as about 0.07 mm to about 0.20 mm, or about 0.10 mm to about 0.15 mm. In some preferred variations, the maximum transverse dimension may be about 0.07 mm to about 0.15 mm, such as about 0.08 mm to about 0.12 mm. In other variations, each fluid passage may comprise a maximum transverse dimension (e.g., width or diameter) of about 0.025 mm to about 0.5 mm, such as about 0.03 mm to about 0.4 mm, about 0.04 mm to about 0.3 mm, or about 0.05 mm to about 0.20 mm.

The curvature of the surfaces defining the passages may be characterized by a radius of curvature (ROC) selected to maintain smooth flow profiles and to minimize turbulence-inducing transitions. For example, an ROC of the surfaces may be about 0.01 mm to about 5 mm, such as about 0.05 mm to about 2 mm, about 0.1 mm to about 1 mm, or about 0.2 mm to about 0.8 mm. In some preferred variations, the radius of curvature of the concave surfaces adjacent the passages may be about 0.1 mm to about 0.4 mm.

An axial dimension (e.g., length) of each passage may further be selected to support laminar or near-laminar flow. In some variations, the axial passage length may be about 0.5 mm to about 20 mm, such as about 1 mm to about 10 mm, about 2 mm to about 8 mm, or about 2 mm to about 6 mm. In additional variations, the axial passage dimension may be about 0.5 mm to about 5 mm, such as about 1.5 mm to about 3 mm. In some variations, an axial length (e.g., length) of each fluid passage relative to a maximum transverse dimension of the insert electrode may be selected to promote laminar or near-laminar flow. For example, a ratio between the axial passage length and the maximum transverse dimension of the insert electrode may be about 11:1 to about 1:20, such as about 1:2 to about 1:10, about 1:3 to 1:9, about 1:4 to about 1:8, about 1:5 to about 1:7, about 1:2 to about 1:5, about 1:3 to about 1:4, about 1:2, about 1:3, about 1:4, about 1:5, about 1:6, about 1:7, about 1:8, about 1:9, about 1:10, about 1:11, or about 1:12 .. Similarly, an axial length (e.g., length) of each fluid passage relative to a maximum transverse dimension of the outer electrode or the fluid lumen may be selected to promote laminar or near-laminar flow. For example, a ratio between the axial passage length and the maximum transverse dimension that of the outer electrode, and/or the fluid lumen, and/or the shaft may be about 1:1 to about 1:20, such as about 1:2 to about 1:10, about 1:3 to 1:9, about 1:4 to about 1:8, about 1:5 to about 1:7, about 1:2 to about 1:5, about 1:3 to about 1:4, about 1:2, about 1:3, about 1:4, about 1:5, about 1:6, about 1:7, about 1:8, about 1:9, about 1:10, about 1:11, or about 1:12. In some variations, the ratio of the maximum transverse dimension of the insert electrode and a length of one or more of the fluid passages may be about 1:4 to about 1:3.

Suitable combinations of passage curvature, axial length, and maximum transverse dimension may promote laminar exit characteristics and maintain Reynolds numbers below about 2300 at clinically relevant irrigation rates. When combined with smooth wall geometries and the symmetric arrangement of the vertices 1027, the passages may distribute fluid around the distal electrode assembly in a uniform manner, support consistent thermal and electrical conditions, and reduce stagnation regions in which clot formation may otherwise occur.

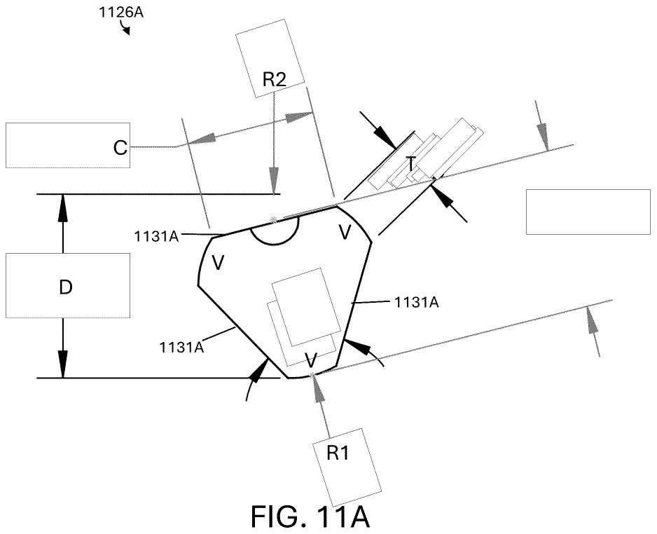

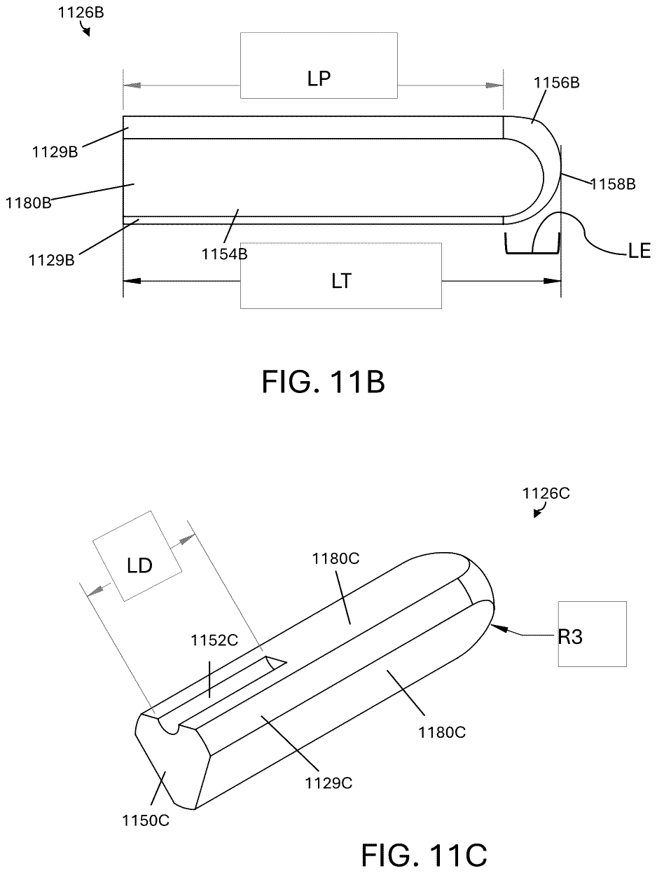

FIG. 11A depicts a transverse cross-sectional view of an insert electrode 1126A having a generally polygonal or triangular transverse profile. The insert electrode 1126A may be configured to be positioned coaxially within an outer electrode (not shown) such that one or more fluid passages are defined between the insert electrode and the surrounding structure.

The insert electrode 1126A may define a transverse dimension C of about 0.25 mm to about 5 mm, about 0.5 mm to about 4 mm, about 0.75 mm to about 3 mm, about 1 mm to about 2 mm, or about 1.5 mm. A longitudinal dimension D defined by the electrode 1126A may represent a perpendicular distance between opposing vertices or opposing sidewall regions and may fall within similar ranges, such as about 0.25 mm to about 5 mm, about 0.5 mm to about 4 mm, about 0.75 mm to about 3 mm, about 1 mm to about 2 mm, or about 1.5 mm. In some variations, the dimensions C and D may be selected to adjust the maximum transverse dimension if fluid outlets and/or electric-field distribution when the insert electrode is nested within the outer electrode.

The profile may include three or more vertices V, each of which may be rounded. The radius of curvature R1 at each vertex may be about 0.1 mm to about 1.0 mm. The rounding of the vertices may mitigate localized electric-field intensification and may support more uniform current delivery.

Adjacent vertices may be joined by sidewalls 1131A, which may be straight, faceted, or curved. An angle defined between adjacent sidewalls may be about 50 degrees to about 90 degrees, such as about 55 degrees to about 70 degrees. Each sidewall may further include a substantially planar portion having a transverse extent T, which may be about 0.1 mm to about 1.0 mm. The sidewalls may also include faceted or linear regions, which may span about 0.5 mm to about 2.0 mm in length or may define heights of about 0.1 mm to about 0.5 mm.

One or more sidewalls may include a convex region C, as depicted. The convex region C may define a recess depth of about 0.2 mm to about 1.0 mm, and may further define a radius of curvature R2 that may be about 0.1 mm to about 0.5 mm. The convex region C may serve as an attachment interface for an electrical conductor or trace and may further enlarge the cross-sectional area of adjacent fluid-flow passages. When the insert electrode 1126A is positioned within an outer electrode, the geometry of the concave region C, the planar segment T, and the rounded vertices V may collectively modulate the maximum transverse dimension of the surrounding fluid passages, thereby supporting laminar flow conditions and reducing clot formation near the distal electrode assembly.

The geometric relationships shown in FIG. 11A—including the transverse dimensions C and D, the vertex radii R1, the concave-region radius R2, the depth S, the planar portion T, and the vertex angles V—may be selected to balance mechanical stability, electrical performance, and controlled fluid-flow behavior within the catheter tip body.

FIG. 11B depicts a side view of an insert electrode 1126B illustrating a proximal portion 1154B and a distal tip portion 1156B that terminates at an apex 1158B. The insert electrode 1126B may comprise an elongate body having a total length LT, which may be about 2 mm to about 5 mm, such as about 2.5 mm to about 4 mm, or about 3 mm. The proximal portion 1154B may define a body having vertices 1129B and sidewalls 1180B. The proximal portion (including the vertices 1129B and sidewalls 1180B) may comprise a length LP, which may be about 0.5 mm to about 5 mm, such as about 0.75 mm to about 4 mm, about 1 mm to about 3 mm, about 1.5 mm to about 3 mm, about 1.5 to about 2.5 mm, about 1 mm, about 1.5 mm, about 2 mm, about 2.5 mm, or about 3 mm.

The distal tip 1156B may comprise a nose or protruded region that transitions into the apex 1158B. The protruded portion may define a length LE that is about 0.25 mm to about 1.5 mm, such as about 0.5 mm to about 1.0 mm. The apex 1158B may have a rounded geometry and may define a radius of curvature R3, which may be about 0.3 mm to about 0.7 mm, such as about 0.45 mm to about 0.55 mm. In some variations, the rounded apex geometry may be configured to reduce tissue trauma during navigation and to modulate electric-field distribution during pulsed-field energy delivery. A ratio between the exposed length LE of the insert electrode and a maximum transverse dimension of the insert electrode 1126B may be about 1:12 to about 1:3, such as about 1:8 to about 1:4. This structural relationship may maintain a compact protrusion while providing sufficient exposed distal surface for energy delivery and fluid interaction.

The longitudinal profile of the insert electrode may define a substantially constant thickness over the length LT, except for tapering or rounding at the distal apex 1158B. In some variations, the proximal portion 1154B may define one or more flat or planar surfaces to facilitate mechanical seating, bonding, or registration within the distal electrode assembly.

FIG. 11C depicts a perspective view of an insert electrode 1126C showing a proximal-facing surface 1150C, an elongate concave or recessed region 1152C, and a rounded distal apex having radius of curvature R3. The recessed region 1152C may function as an attachment or connection feature for an electrical conductor. The recessed region may define a recess depth LD, which may be about 0.2 mm to about 1.0 mm, such as about 0.4 mm to about 0.8 mm, or about 0.6 mm. The recess may extend over about 5% to about 75% of a total length of the insert LT, such as 12% to about 60% of the length LT, about 15% to about 50% of the length LT, or about 20% to about 40% of the length LT.

The recessed region 1152C may comprise a slot, channel, concave trough, or non-planar cutout that accommodates a solder joint, welded joint, conductive adhesive, or other electrical connection point for coupling the insert electrode to a central or offset conductor. The geometry of the recess may be linear, slightly curved, or tapered along its length to improve strain relief and reduce stress concentration at the conductor interface.

The distal apex of the insert may define the same or similar radius of curvature R3 described above with respect to FIG. 11B. In some variations, the curvature of the apex may blend smoothly into adjacent longitudinal sidewalls, which may themselves include straight, faceted, concave, or slightly convex sections depending on the transverse geometry described with respect to FIG. 11A.

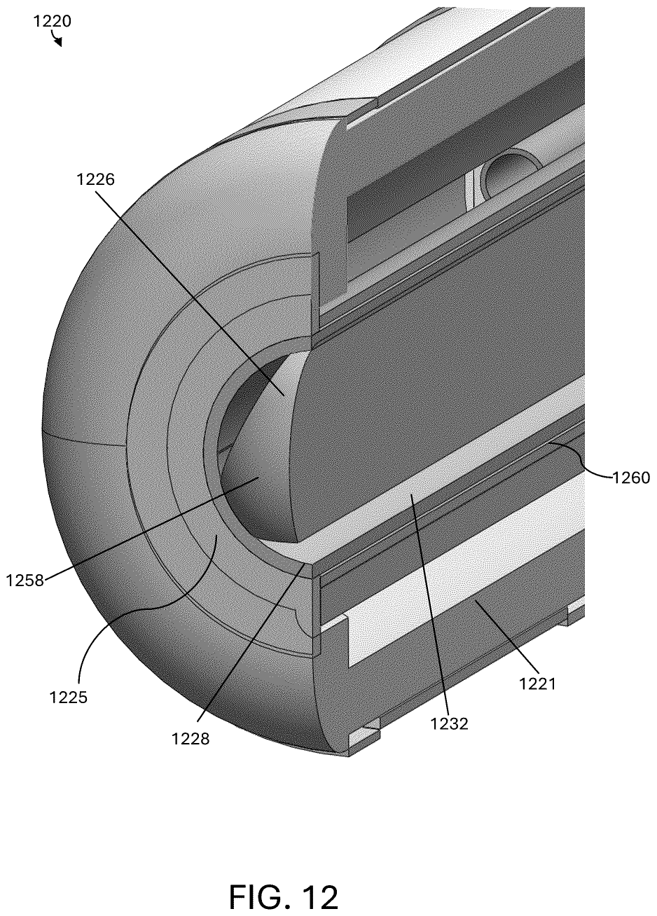

FIG. 12 depicts a partial perspective cutaway view of a tip body assembly 1220 illustrating an exemplary relationship between an outer electrode 1228, an insert electrode 1226, and surrounding insulative and structural components. In this view, a portion of the tip body 1221 has been removed to reveal an internal cavity 1260 configured to receive the distal electrode assembly 1225. The outer electrode 1228 may define a generally cylindrical or partially rounded internal surface that at least partially surrounds the insert electrode 1226 along a coaxial arrangement within the cavity 1260.

As shown, the insert electrode 1226 may be positioned distally within the cavity 1260 such that its distal apex 1258 aligns with or protrudes slightly beyond a rounded distal contour of the outer electrode 1228. The outer electrode 1228 may define a lumen 1232 sized to accommodate the insert's transverse geometry as described with respect to FIGS. 11A-11C. This geometry may maintain one or more interstitial fluid-flow passages between the insert and the inner surface of the outer electrode, consistent with the fluid paths shown and described in FIG. 10.

The tip body 1221 may include one or more structural layers, reinforcement bands, or polymeric sleeves that may hold the insert and outer electrodes in position relative to each other. The tip body 1221 may further define one or more lumens or channels configured to receive conductors routed from the shaft toward the distal electrode assembly 1225 or proximal electrode(s) (not shown), as described with respect to FIG. 8. In some variations, a solder joint, mechanical interference fit, and/or an adhesive layer may couple the insert electrode 1126 to the tip body 1221 or to an inner surface of the outer electrode lumen 1232.

### Alternative Variations

### Distal Electrode Assembly Variations

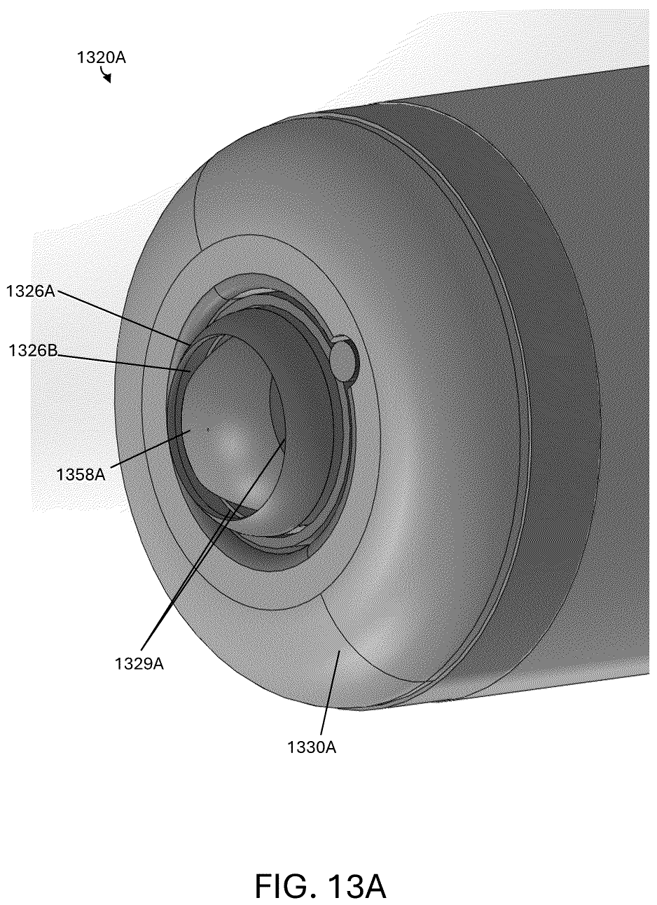

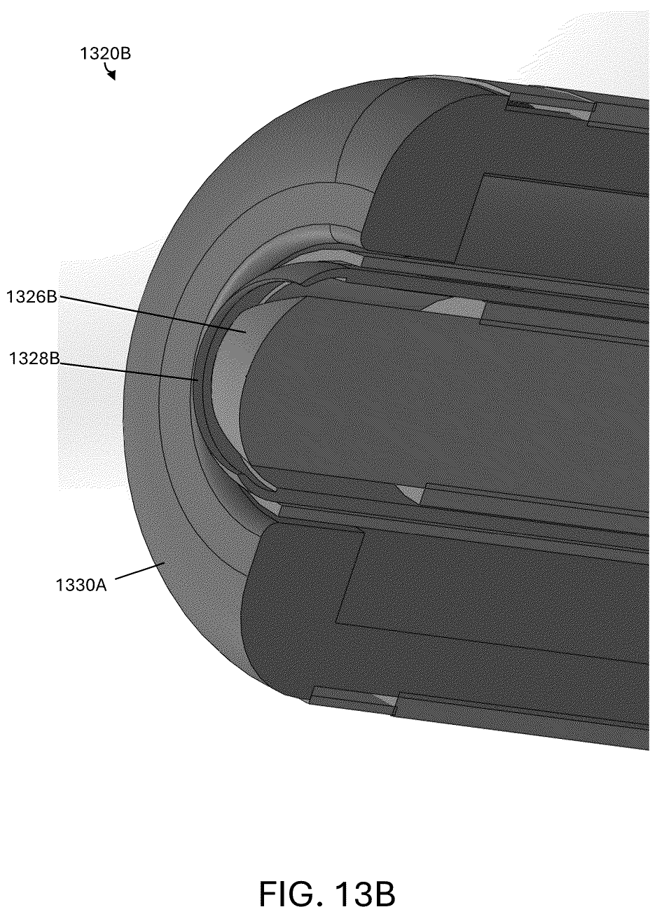

Additional and alternative variations of the distal electrode assembly are described below with reference to FIGS. 13A-20

FIGS. 13A and 13B depict a variation of tip body 1320 that includes an outer electrode 1328 having a distally curved, crowned, or partially enclosed distal region 1328a extending around a distal face 1330 of the tip body. In this variation, the outer electrode comprises a substantially cylindrical proximal portion 1328b that transitions distally into a curved or tapered segment 1328a. This distal transition region may be formed by crimping, bending, stamping, or tapering the distal end of the outer electrode such that the distal opening of the electrode is partially closed. The curvature may be circumferential or segmental and may be defined by a radius selected to redirect fluid flow and to control the effective distal aperture of the outer electrode.

The curved distal region 1328a may extend distally beyond, flush with, or slightly recessed relative to the distal-most surface of the tip body 1320, and may partially enclose the distal region of the tip body to create a deflecting surface for irrigation fluid routed through the fluid lumen. As shown, fluid exiting the shaft may encounter the inner surface 1328c of the curved region, causing the fluid stream to be redirected laterally or proximally along the contours of the tip body rather than exiting directly forward along the catheter's longitudinal axis. In some variations, this redirected flow profile may reduce high-velocity jetting from the distal end and may promote circumferential dispersion of irrigation fluid around the distal electrode assembly.

The insert electrode 1326 is shown recessed within an internal recess 1320a of the tip body, with its distal end 1326a positioned proximally relative to the curved distal region 1328a of the outer electrode. This recessed placement may shield the insert electrode from direct forward flow, reduce the likelihood of axial spray from the fluid lumen, and promote redirection of fluid along the external surfaces of the tip body. The recessed configuration may also partially shield the insert electrode during pulsed electric field activation, limiting direct tissue contact with the distalmost conductive surface and contributing to a more controlled electric-field distribution.

Building on the distally curved or partially enclosed configurations of FIGS. 13A-13B, FIGS. 14-22 illustrate additional variations in which the tip body is partially or fully closed to shape fluid release, protect the internal insert, and/or influence the emitted electric field distribution.

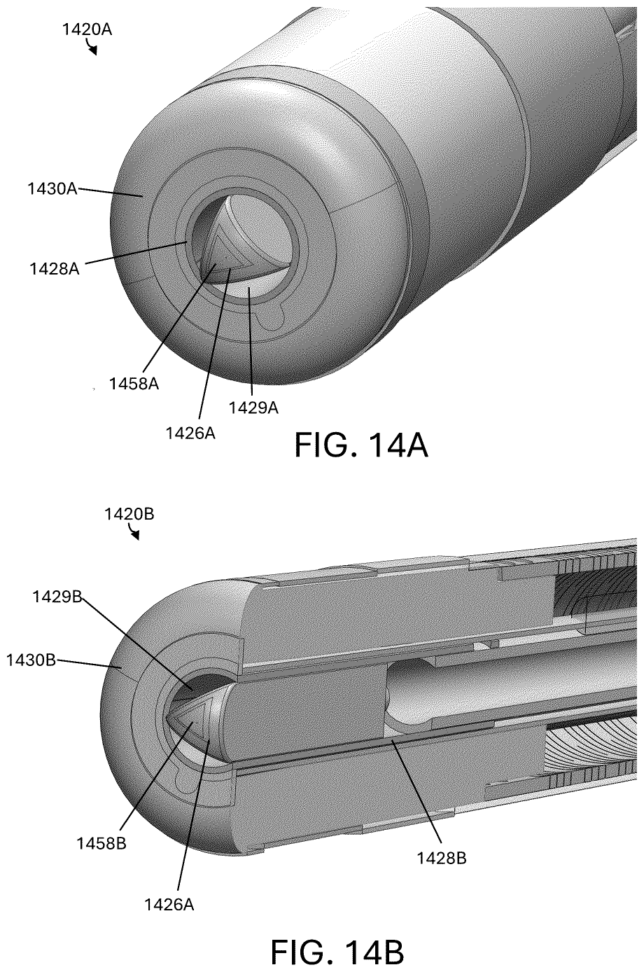

FIGS. 14A and 14B depict a variation of a distal electrode assembly in which the insert electrode 1426A/1426B is distally exposed relative to the distal face 1430A/1430B of the tip body 1420A/1420B. In this variation, the insert electrode 1426A/1426B comprises a three-sided, generally triangular distal geometry, with three planar or slightly curved side faces converging at a pointed or rounded apex 1458A/1458B. The apex 1458A/1458B extends distally beyond the surrounding distal face 1430A/1430B, forming a pronounced distal tip that serves as the primary energy-delivery surface.

The outer electrode 1428A/1428B defines a circular distal opening through which the triangular insert electrode 1426A/1426B passes. The inner circumferential surface of the outer electrode 1428A/1428B is radially spaced from the three side faces of the insert electrode, creating three discrete fluid passages 1429A/1429B. Each passage is crescent-shaped or arcuate in transverse cross-section, defined between a respective vertex region of the triangular insert and the opposed curved inner wall of the outer electrode. These passages 1429A/1429B open directly to the distal face 1430A/1430B surrounding the insert electrode apex, allowing irrigation fluid to exit radially or obliquely around the protruding triangular tip.

As shown in FIG. 14B, the proximal portion of the insert electrode 1426B extends inward from the apex as a solid, tapered conductive body that transitions proximally into a cylindrical or slightly conical form. No grooves, channels, ribs, or perforations are present in this variation; fluid flow is governed solely by the three interstitial passages 1429B formed between the stationary geometry of the insert and outer electrodes. The passages extend along a short axial length between the proximal interior region and the distal outlets, providing a smooth-walled, uninterrupted flow path configured to promote laminar or near-laminar conditions at clinically relevant flow rates.

The distal face 1430A/1430B of the tip body has a rounded, dome-like contour, and the outer electrode 1428A/1428B seats within this rounded geometry, forming a continuous transition between the body of the catheter and the distal opening. The three outlet regions surrounding the triangular insert electrode produce a three-fold symmetry in the distal flow pattern and may generate a correspondingly symmetric electric-field distribution around the protruding apex 1458A/1458B.

The passage walls include smooth, continuous surfaces with no protrusions or grooves along the insert, allowing fluid to transition from the proximal lumen into the distal openings without abrupt turns or disruptions.

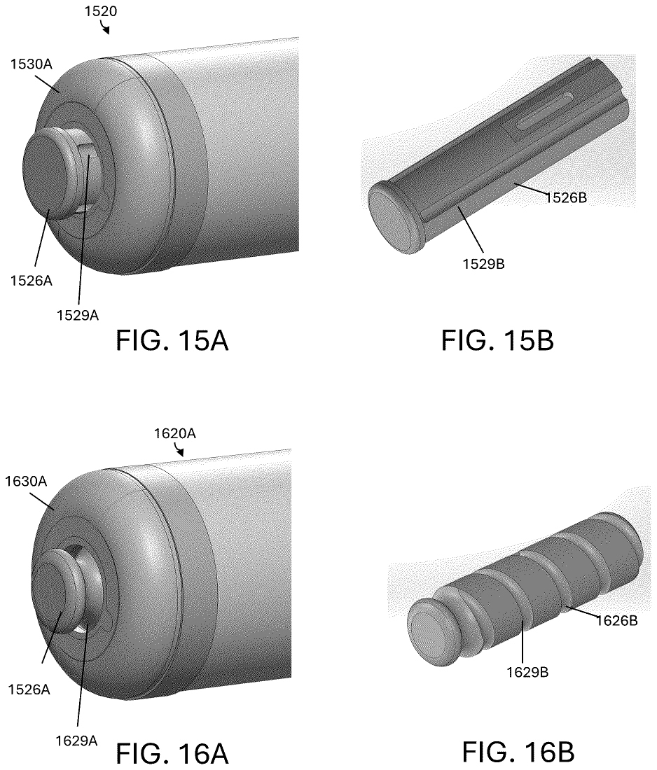

FIGS. 15A and 15B illustrate a variation of the distal electrode assembly in which the insert electrode 1526A, 1526B comprises an elongated, generally cylindrical conductive body that protrudes distally beyond the distal face 1530A of the tip body. As shown, the insert electrode includes a series of longitudinal fluid-flow channels 1529A, 1529B formed directly into the outer surface of the conductive body. Each channel extends along substantially the full axial length of the insert electrode from a proximal region to the distal apex 1558B, maintaining a consistent transverse profile along its course. In some variations, the channels may have rounded or arcuate inner surfaces and may be dimensioned to promote smooth, uninterrupted flow without abrupt changes in cross-section.

The channels 1529A, 1529B open distally at the distal apex 1558B such that the insert electrode defines multiple discrete distal outlets positioned circumferentially around the distal tip. Because the channels run continuously along the length of the insert, fluid routed through the fluid lumen of the catheter may enter the channels proximally, travel distally along each channel, and exit through the distal outlets as multiple parallel flow streams. This longitudinal channel structure differs from variations in which fluid passages are defined solely between a non-circular insert electrode and an opposing inner surface of an outer electrode. Here, the fluid passages are integrated into the insert electrode itself, allowing the distal tip to define its own set of flow conduits independent of surrounding geometry.

The insert electrode shown in FIGS. 15A and 15B further exhibits a bottle-neck or stepped profile in which a narrower proximal portion transitions into a wider distal portion that defines the distal apex 1558B. The transition may be curved, tapered, or radiused to maintain mechanical robustness while reducing abrupt changes in transverse dimension. This distal enlargement increases the exposed conductive surface area and produces a more pronounced distal leading edge during pulsed electric field delivery. When combined with the distributed outlets defined by channels 1529A, 1529B, this geometry may promote a more spatially uniform fluid envelope around the distal tip and may influence the resulting electrical field distribution by reducing stagnation regions and directing conductive cooling fluid over a larger region.

The annular gap between the outer surface of the protruding insert electrode 1526A and the distal face 1530A further defines an additional circumferential fluid-release region. This gap may interact with the longitudinal channels to create a combined outflow pattern in which fluid exits both axially and radially around the distal tip. The distal-facing configuration of this embodiment is therefore characterized by a protruding conductive structure with integrated channels, a widened distal apex, and multiple distributed outlets that extend distally beyond the surrounding tip body. This arrangement provides a unique combination of protrusion, channel-based flow delivery, and distal tip geometry relative to the more recessed or enveloped configurations described elsewhere in this specification.

FIGS. 16A and 16B depict a variation in which the insert electrode 1526 comprises a stepped or ribbed conductive structure positioned within a circular opening 1528a of outer electrode 1528. As seen in FIG. 16B, the insert electrode includes a series of circumferential ribs 1526b spaced along its longitudinal length to create alternating raised lands and recessed grooves. These ribs may be formed integrally with the insert electrode or may be machined into its outer surface. The stepped geometry may increase the outer diameter at discrete axial intervals, producing a profile that engages complementary internal features 1520d within tip body 1520, as illustrated in the sectional view of the tip region. The distal face 1526a of the insert electrode may be substantially planar or slightly domed and may be arranged flush with, or recessed within, the distal opening of the outer electrode. The ribbed configuration may confine distal fluid release to the annular space surrounding the distal face and may define internal fluid-expansion regions between adjacent ribs when irrigation fluid is routed distally through the shaft. This variation maintains a predominantly closed-face distal geometry while providing a defined conductive surface at the distal end of the insert electrode.

FIGS. 16A and 16B illustrate a variation in which the insert electrode 1626 includes a continuous helical fluid passage 1629 extending along substantially the entire length of the insert electrode. As shown in FIG. 16B, the outer surface of the insert electrode 1626B defines a smooth, spiraling recess that wraps circumferentially around the electrode in a screw-like pattern. Adjacent turns of the helical recess form raised circumferential land regions that collectively give the insert body a ribbed or stepped external appearance. However, the recess itself is a single uninterrupted channel, rather than a series of separate axial grooves.

The helical fluid passage 1629B may begin at or near a proximal region of the insert electrode and may extend distally toward the distal apex 1658A. In FIG. 16A, the distal tip of the helical passage 1629A remains open at the distal face 1630A of the tip body 1620A, such that the helical channel provides a continuous fluid pathway from the fluid lumen within the tip body to one or more distal exit openings positioned at or immediately adjacent to the distal apex 1658A.

The helical geometry may define a substantially constant transverse dimension along its length or may taper slightly as the channel advances distally. In some variations, the helical passage 1629 may have a maximum transverse dimension of about 0.05 mm to about 0.30 mm, such as about 0.07 mm to about 0.20 mm or about 0.10 mm to about 0.15 mm, and the raised land regions between adjacent helical turns may maintain a smooth outer contour configured to reduce stagnation and to maintain laminar or near-laminar flow. The curvature of the helical passage may be further defined by a radius of curvature of about 0.1 mm to about 5 mm, such as about 0.3 mm to about 2 mm or about 0.5 mm to about 1.0 mm, which may be selected to balance manufacturability, flow conductance, and maintenance of laminar flow at irrigation rates of about 1-5 mL/min.

When the insert electrode 1626A is positioned within the tip body 1620A, the raised helical lands may interface closely with the opposing inner surface of the outer electrode 1628A, defining a helical interstitial region that forms the effective fluid passage along which irrigation fluid is routed. The continuous spiral pathway may increase the effective flow length, limit direct axial jetting, and distribute fluid uniformly around the perimeter of the distal electrode assembly. This extended helical flow path may further promote cooling, minimize clot-forming stagnation regions, and provide a controlled impedance environment for pulsed-field energy delivery.

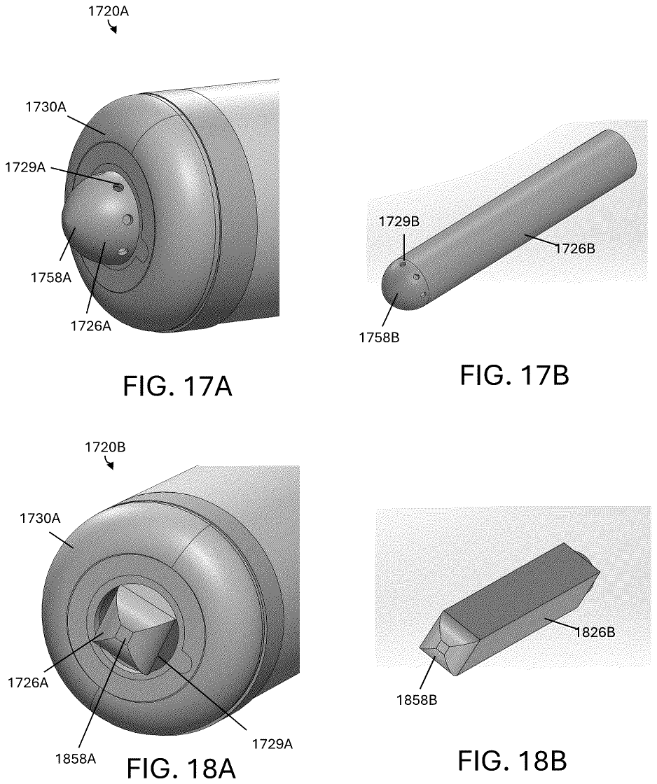

FIGS. 17A and 17B illustrate a variation comprising a hemispherical insert electrode 1726 positioned distally of the distal face 1730A of the tip body 1720A. The insert electrode includes a substantially cylindrical proximal portion that transitions into a smooth, curved distal hemisphere. The distal apex 1758A defines the outermost point of the hemisphere, and the curvature transitions continuously along the outer surface without facets, ribs, or abrupt changes in cross-section.

As shown in FIG. 17B, the distal hemispherical region includes multiple discrete fluid-passage apertures 1729B formed through the curved distal wall. These apertures are distributed circumferentially around the hemispherical surface near the apex 1758B. Each opening is formed as an individual circular perforation, and the apertures are not connected by channels or grooves along the proximal portion of the insert. No fluid-flow passages extend longitudinally along the cylindrical shaft of the insert electrode; instead, all fluid outlets are located within the distal hemispherical region.

In FIG. 17A, the proximal cylindrical portion of the insert electrode 1726A fits within an opening defined at the distal face 1730A of the tip body 1720A, while the hemispherical region projects distally beyond the distal face. The radial spacing between the proximal cylindrical section of the insert and the inner surface of the tip body may define one or more annular or arcuate fluid regions proximal to the hemispherical outlets. The hemispherical surface may have a radius of curvature of about 0.2 mm to about 5 mm, such as about 0.5 mm to about 2 mm or about 0.8 mm to about 1.5 mm, and the axial height of the hemispherical region may be about 0.1 mm to about 3 mm, such as about 0.3 mm to about 1.5 mm.

The cross-section of the proximal portion of the insert electrode is circular, with the distal hemisphere forming a continuous, rounded termination. The relative positioning of the distal apertures 1729A around the hemispherical surface defines peripheral fluid-flow pathways that exit radially or diagonally from the distal region.

FIGS. 18A and 18B depict a variation in which the insert electrode 1826 has a polygonal cross-section and a faceted distal apex 1858. As shown in FIG. 18B, the insert electrode comprises a series of planar side surfaces that converge toward the apex at the distal end. These planar facets meet at defined edges, forming a sharply tapered pyramidal tip. The transition between the polygonal proximal section and the distal apex region occurs over a tapered axial segment that narrows as it approaches the apex.

In FIG. 18A, the insert electrode 1726A extends distally beyond the distal face 1830 of the tip body 1820B. The distal face defines an opening that closely conforms to the polygonal profile of the insert. Because the insert electrode has multiple vertices, discrete fluid-flow passages 1729A are defined at the corner regions between each vertex and the opposing inner surface of the distal opening. The fluid passages are therefore shaped by the geometric relationship between the flat facets of the insert and the surrounding curved inner surface of the tip body opening.

The fluid passages 1829B extend proximally from the distal face 1830B along the length of the polygonal shaft region. Each passage occupies a wedge-or crescent-shaped space located at each vertex of the polygonal insert. The cross-sectional shape of the passages remains largely dependent on the polygonal geometry of the insert, while the distal apex region 1858A remains solid and free of perforations. Fluid exits the distal region through gaps positioned around the apex, not through apertures in the apex itself.

The axial taper length between the polygonal shaft and the pyramidal apex may range from about 0.1 mm to about 3 mm, such as about 0.3 mm to about 1.5 mm. The facets forming the apex may define taper angles of about 10 degrees to about 60 degrees relative to the longitudinal axis. A radius of curvature may be used to describe optional rounding at the facet intersections; such a radius may be about 0 mm (sharp edge) to about 0.5 mm, such as about 0.1 mm to about 0.3 mm.

The polygonal cross-section of the insert electrode may be triangular, quadrilateral, or another multi-sided geometry, and may include three or more vertices that define the number and positioning of fluid-passage regions 1829A.

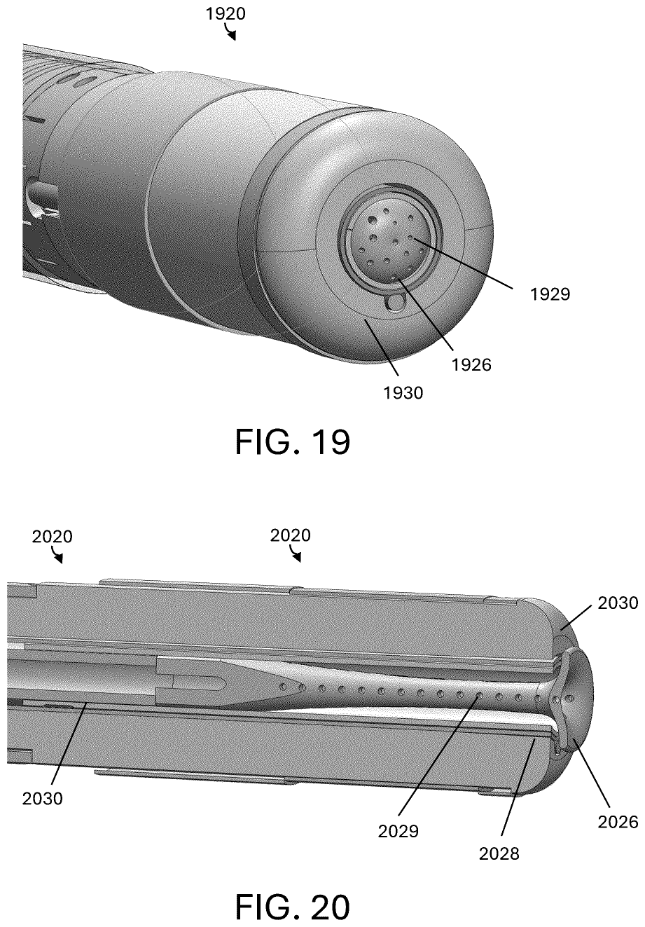

FIG. 19 illustrates a variation in which the insert electrode 1926 comprises a rounded distal region projecting distally from the distal face 1930 of the tip body 1920. The distal portion of the insert electrode 1926 forms a generally bulbous or spheroid-cap geometry, with a smooth continuous curvature extending from the cylindrical proximal portion to the distal apex 1958.

A plurality of fluid-passage apertures 1929 are formed through the distal curved wall of the insert electrode. The apertures are arranged across the rounded distal surface and appear distributed in a pattern surrounding the apex, with openings positioned at multiple circumferential locations. Each aperture is a discrete perforation of generally circular cross-section.

The proximal cylindrical portion of the insert electrode 1926 is received within a circular opening formed at the distal face 1930 of the tip body, while the distal tip of the insert electrode 1926 protrudes distally beyond the distal face 1930.

FIG. 20 depicts a variation in which the insert electrode 2026 includes a proximal portion that is at least partially surrounded by a fluid lumen 2030 within the tip body 2020. The proximal region of the insert electrode includes one or more substantially planar side surfaces and transitions distally into a tapered intermediate section. The taper narrows along the longitudinal axis before expanding outward again to form a rounded distal region.

The insert electrode 2026 includes a plurality of fluid-passage apertures 2029 arranged along the tapered distal section. These apertures are formed as discrete perforations distributed longitudinally along the narrowing portion of the electrode body. The apertures are generally circular and aligned along multiple axial rows. The proximal cylindrical or planar-sided region of the insert electrode lacks openings, with the perforations beginning only after the taper initiates. The distal apex region is solid and free of perforations, with all fluid exits located along the tapered intermediate segments.

The distal face 2030 of the tip body surrounds the proximal portion of the insert electrode and defines an opening through which the electrode extends. Proximal to the tapered region, the insert electrode may be positioned within or adjacent to the fluid lumen 2030 such that fluid may surround at least a portion of the proximal electrode body. Beyond this interface, the tapered distal portion extends distally of the distal face 2030 and is exposed outside of the tip body 2020.

### Laminar Flow Considerations

In some variations, the distal electrode assemblies described herein may be configured such that fluid exiting the distal outlets forms a laminar or near-laminar flow pattern along the tip body. Laminar flow may be preferred for cooling, reduction of clot formation, and electrical current transfer. In some variations, the term ‘near-laminar’ may refer to flow regimes in which Reynolds numbers remain within about 10% to about 50% of the fully laminar regime observed within the fluid passages described herein (e.g., Reynolds numbers less than about 3450, such as those less than about 2300 or within a range of about 200 to about 1100 at irrigation flow rates of about 1 to about 5 mL/min).

From a cooling standpoint, laminar flow may promote a uniform, consistent fluid layer over the distal electrode assembly and adjacent surfaces. By maintaining a stable velocity profile and minimizing recirculation zones, laminar flow may allow heat to be carried away from the electrodes in a predictable manner. In contrast, turbulent flow may create localized stagnation regions and recirculating loops of heated fluid that remain in contact with the electrodes, which may reduce effective cooling by repeatedly exposing the fluid to elevated electrode temperatures.

Laminar flow may also support reduction of clot formation at or near the tip body. When saline or other biocompatible fluid is flushed through the distal electrode assembly, the laminar velocity profile through the outlet passages may generate shear stresses on blood or blood products that contact the flowing fluid. Because the linear velocity of the fluid is higher in the center of the outlet channels than near the channel walls, a velocity gradient may be established that produces shear forces which may promote dissolution or disruption of nascent clots in or around the tip body.

Electrical current transfer between the distal electrode assembly and surrounding tissue may further benefit from laminar flow conditions. As the cooling fluid exits the distal outlets and mixes with blood, differences in electrical conductivity between the saline and blood may influence the effective field distribution at the electrodes. A laminar flow regime may maintain a more consistent layer of saline at the electrode surface, thereby providing a more uniform and predictable conductive path for energy delivery. In contrast, turbulent flow may promote unpredictable mixing between blood and saline, creating spatially variable conductivity and less consistent electrical coupling.

In some variations, the distal outlet geometry and operating flow rates may be selected such that the Reynolds number (Re) for flow within the outlet passage remains below about 2,300, corresponding to laminar-dominated flow for internal pipe flow. For a given fluid viscosity, the Reynolds number may be reduced by decreasing the maximum transverse dimension of the outlet passage and/or by selecting lower flow velocities, while still providing sufficient volumetric flow for cooling and clot reduction.

### II. Method

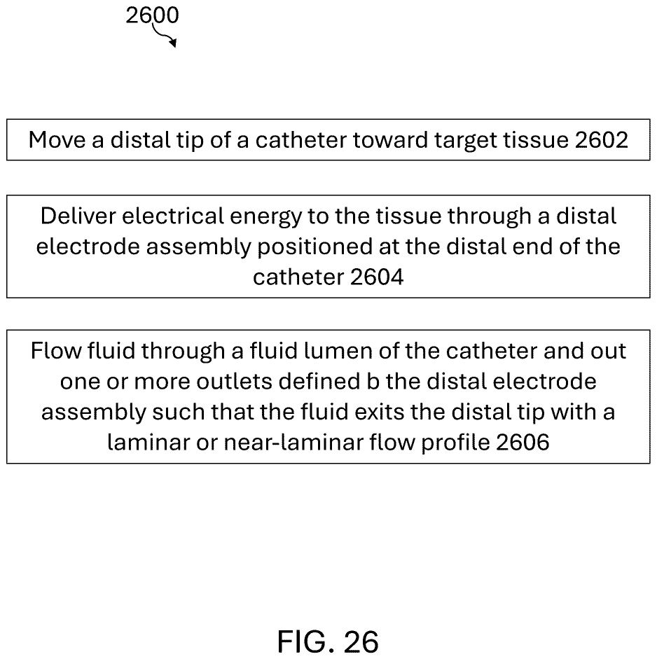

FIG. 26 illustrates an exemplary method 2600 for operating a catheter having a distal electrode assembly as described herein. Although illustrated as a sequence of discrete operations, the steps of the method may be performed in any suitable order or concurrently, and one or more steps may be omitted, repeated, or combined. The method 2600 may be carried out manually by an operator or partially or fully automated using a console that controls the catheter, energy source system, and fluid source. The method 2600 may be performed using any of the tip body geometries, fluid outlets, or electrode configurations described herein.

At step 2602, a tip body of a catheter may be advanced to a target site within a patient. The catheter may include a shaft having one or more fluid lumens in fluid communication with a distal electrode assembly comprising an insert electrode positioned within, or coaxially relative to, an outer electrode, with fluid passages defined therebetween. The coaxial or nested electrode configurations described herein may provide substantially uniform electric-field distribution regardless of approach angle, reducing orientation dependencies encountered in conventional bipolar systems.

At step 2604, electrical energy may be delivered between the distal electrode assembly and one or more additional electrodes. For pulsed-field ablation, electrical energy may be delivered as short-duration pulses configured to induce reversible or irreversible electroporation. Each pulse of the waveform may comprise a voltage of between about 100 V and about 60 kV, between about 100 V and about 30 kV, between about 100 V and about 20 kV, between about 100 V and about 10 kV, between about 100 V and about 8 kV, between about 100 V and about 6 kV, between about 100 V and about 4 kV, between about 100 V and about 2 kV, between about 100 V and about 1 kV, between about 1 kV and about 60 kV, between about 1 kV and about 30 kV, between about 1 kV and about 20 kV, between about 1 kV and about 10 kV, between about 1 k V and about 8 kV, between about 1 kV and about 6 kV, between about 1 kV and about 4 kV, between about 1 kV and about 2 kV, between about 2 kV and about 20 kV, between about 4 kV and about 20 kV, between about 6 kV and about 20 kV, between about 8 kV and about 20 kV, between about 10 kV and about 20 kV, between about 10 kV and about 30 kV, between about 10 kV and about 40 kV, between about 10 kV and about 50 kV, between about 10 kV and about 60 kV, between about 12 kV and about 20 kV, between about 12 kV and about 25 kV, between about 12 kV and about 30 kV, between about 12 kV and about 40 kV, between about 15 kV and about 20 kV, between about 15 kV and about 25 kV, between about 15 kV and about 30 kV, between about 20 kV and about 40 kV, between about 20 kV and about 50 kV, between about 20 kV and about 60 kV, about 7 kV to about 25 kV, about 8 kV to about 24 kV, about 9 kV to about 23 kV, about 10 kV to about 22 kV, about 11 kV to about 20 kV, about 12 kV to about 19 kV, about 5 kV, about 10 kV, about 15 kV, and about 20 kV. Pulse amplitude, pulse duration, repetition rate, and waveform shape may vary depending on the desired clinical effect and the operating parameters of the energy source. Monophasic, biphasic, monopolar, or bipolar waveforms may be used. Monophasic, biphasic, monopolar, or bipolar waveforms may be used. During pulsed electric field energy delivery, laminar flow through the passages of the distal electrode assembly may maintain a consistent conductive interface at the electrode surfaces and may reduce variations in local field intensity associated with irregular mixing at the fluid-blood boundary The maintained laminar flow may enable higher power delivery while maintaining tissue interface temperatures below about 80° C., reducing the likelihood of steam pops, char formation, or impedance rise.

In some variations, the method 2600 may include adjusting one or more waveform parameters, including, for example, pulse delivery timing, may be adjusted based on real-time feedback. Sensors within or near the distal electrode assembly may monitor temperature, impedance, contact force, and/or the like. The controller may adjust (e.g., increase or decrease) or maintain fluid flow rates within a predetermined range based on received sensor data so that laminar flow through the passages of the distal electrode assembly is maintained (maintaining Reynolds number below 2300, as shown in FIG. 27) while ensuring adequate cooling and clot prevention or mitigation. For example, if tissue temperature approaches about 75° C. during energy delivery, the flow rate may be increased from about 2 mL/min to about 4 mL/min while maintaining a Reynolds number below about 1000.

At step 2606, fluid, such as normal saline, heparinized saline (e.g., containing about 1-10 units/mL heparin), half-normal saline, or other biocompatible irrigation solution, may be flowed through the fluid lumen toward one or more outlets formed in or adjacent to the distal electrode assembly. In some variations, the fluid may flow through a lumen defined by an integrated tubular conductor that provides both electrical connectivity to the distal electrode assembly and structural support for the fluid pathway, as illustrated in FIGS. 23-25. The fluid may flow through passages defined between a triangular or polygonal insert electrode (as illustrated in FIGS. 9-12) and an inner boundary of the outer electrode. These passages may have maximum transverse dimensions of about 0.05 mm to about 0.15 mm, which may promote laminar or near-laminar flow at flow rates of about 1 mL/min to about 5 mL/min, as shown in FIG. 27. Laminar flow (Reynolds number <2300) may provide consistent velocity profiles and predictable shear distribution, may limit recirculation of heated fluid, and may promote directional heat removal from the electrode surfaces. The resulting shear stresses may discourage platelet aggregation or fibrin deposition, thereby reducing clot formation. The uniform fluid layer may provide a stable electrical interface on the electrode surfaces, reducing conductivity fluctuations that could arise from turbulent blood-fluid mixing and improving both ablation uniformity and sensing stability.

In some variations, the method may include interleaved ablation and sensing phases. During sensing phases, the coaxial or nested electrode configuration with substantially uniform radial spacing may reduce directional sensitivity and bipolar blindness compared to conventional side-by-side electrode pairs. The insert electrode may detect near-field activation signals while the outer electrode may capture more regional or far-field components. The maintained radial separation may provide predictable bipolar signal characteristics regardless of wavefront approach angle. During sensing phases, the continued laminar flow may maintain a stable electrical interface, reduce baseline drift, prevent intermittent blood contact with the electrodes, and reduce noise artifacts. Flow at about 1 mL/min to about 2 mL/min may be sufficient to maintain a clear electrode-tissue interface between ablation applications and may clear cellular debris while maintaining adequate tissue contact for immediate electrogram assessment.

The methods enabled by the disclosed catheter design may provide several advantages over conventional ablation approaches. The laminar flow conditions may reduce procedure time by minimizing clot formation and reducing the need for catheter exchanges. The substantially orientation-independent field distribution may reduce the skill dependency of lesion creation. The integrated conductor-fluid pathway may reduce catheter diameter, improving access to small chambers or vessels. The stable electrical interface provided by laminar flow may enhance both ablation predictability and mapping accuracy, potentially reducing overall procedure time and improving acute and long-term success rates.

### EXAMPLES

### Example 1: Laminar Flow Characterization

An illustrative analysis of laminar flow conditions for a representative distal outlet configuration is summarized in the table depicted in FIG. 27. For each of several flow rates between about 1 mL/min and about 5 mL/min, the corresponding average fluid velocity (v), Reynolds number (Re), and fluid passage length were calculated for a distal outlet having a selected maximum transverse dimension. A critical diameter Dcrit associated with a Reynolds number of 2,300 was also determined for comparison.

As shown in FIG. 27, for outlet flow rates between about 1 mL/min and about 5 mL/min, the computed Reynolds numbers ranged from about 200 to about 1,000, all below the 2,300 threshold typically associated with transition to turbulence in internal flow. The corresponding entrance length remained less than about 5 mm (about 0.2 in), which is comparable to or shorter than the axial length of the distal outlet passages. For each flow rate, the calculated critical diameter Dcrit associated with Re=2,300 was greater than the outlet diameter used in the current configuration (highlighted values), confirming that the design operates in a laminar-dominated regime across the examined flow range.

These calculations indicate that, for the selected outlet maximum transverse dimensions and flow rates, fluid exiting the distal electrode assembly is expected to exhibit laminar or near-laminar flow throughout the relevant portion of the distal passages. In practice, this laminar flow pattern may support uniform cooling, promote shear-mediated disruption of clot formation, and provide a stable fluid interface for consistent electrical coupling during ablation, stimulation, or sensing procedures as described above.

Throughout this application, the term “about” is used to indicate that a value includes the inherent variation of error for the device or the method being employed to determine the value, or the variation that exists among the samples being measured. Unless otherwise stated or otherwise evident from the context, the term “about” means within 10% above or below the reported numerical value (except where such number would exceed 100% of a possible value or go below 0%). When used in conjunction with a range or series of values, the term “about” applies to the endpoints of the range or each of the values enumerated in the series, unless otherwise indicated. As used in this application, the terms “about” and “about” are used as equivalents.

While certain variations are described and illustrated herein, those of ordinary skill in the art will readily envision a variety of other means and/or structures for performing the function and/or obtaining the results and/or one or more of the advantages described herein, and each of such variations and/or modifications is deemed to be within the scope of the inventive variations described herein. More generally, those skilled in the art will readily appreciate that all parameters, dimensions, materials, and configurations described herein are meant to be exemplary and that the actual parameters, dimensions, materials, and/or configurations will depend upon the specific application or applications for which the inventive teachings is/are used. Those skilled in the art will recognize or be able to ascertain using no more than routine experimentation, many equivalents to the specific inventive variations described herein. It is, therefore, to be understood that the foregoing variations are presented by way of example only and that, within the scope of the appended claims and equivalents thereto; inventive variations may be practiced otherwise than as specifically described and claimed. Inventive variations of the present disclosure are directed to each individual feature and/or method described herein. In addition, any combination of two or more such features and/or methods, if such features and/or methods are not mutually inconsistent, is included within the inventive scope of the present disclosure.

1. An ablation catheter, comprising:
an elongate body having a fluid lumen extending therethrough; and
a distal electrode assembly coupled to the elongate body and comprising:
an insert electrode having a non-circular cross-sectional shape with at least three vertices; and
an outer electrode at least partially surrounding the insert electrode and electrically coupled thereto, the outer electrode positioned at or adjacent to a distal end surface of a tip of the catheter,
wherein spaces between the vertices of the insert electrode and an inner surface of the outer electrode define fluid passages in fluid communication with the fluid lumen.

2. The ablation catheter of claim 1, wherein the insert electrode protrudes distally an exposed length beyond the distal end surface of the tip of the catheter.

3. The ablation catheter of claim 2, wherein the exposed length of the insert electrode is about 0.5 mm to about 1 mm.

4. The ablation catheter of claim 2, wherein a ratio between the exposed length of the insert electrode and a diameter of the elongate body of the catheter is about 1:8 to about 1:4.

5. The ablation catheter of claim 2, wherein a ratio between the exposed length of the insert electrode and a maximum transverse dimension of the insert electrode is configured to promote a substantially symmetric electrical field about a longitudinal axis of the elongate body.

6. The ablation catheter of claim 1, wherein a distal end surface of the outer electrode is substantially flush with the distal end surface of the tip of the catheter.

7. The ablation catheter of claim 1, wherein the outer electrode comprises a circular cross-sectional shape.

8. The ablation catheter of claim 1, wherein the outer electrode surrounds a proximal portion of the insert electrode disposed within a lumen in the tip of the catheter.

9. The ablation catheter of claim 8 further comprising a tip body, wherein the lumen in the tip of the catheter is a lumen in the tip body.

10. The ablation catheter of claim 1, wherein each vertex forms an interference fit with a corresponding portion of the outer electrode.

11. The ablation catheter of claim 1, wherein each of the fluid passages terminates at an outlet positioned around the insert electrode at the distal end surface of the tip of the catheter, and wherein at least one outlet is oriented to direct fluid flow radially outward from or radially inward toward a central longitudinal axis of the elongate body.

12. The ablation catheter of claim 1, wherein the vertices of the insert electrode are spaced at substantially equal angular intervals around a central longitudinal axis of the insert electrode.

13. The ablation catheter of claim 1, wherein the insert electrode further comprises arcuate sidewalls extending between adjacent vertices, and wherein each fluid passage is formed between one of the arcuate sidewalls and an inner surface of the outer electrode.

14. The ablation catheter of claim 1, wherein the insert electrode comprises a triangular cross-sectional shape.

15. The ablation catheter of claim 1, wherein the insert electrode divides fluid from the fluid lumen into three discrete fluid passages.

16. The ablation catheter of claim 1, wherein the distal electrode assembly is configured to provide laminar fluid flow through each of the fluid passages.

17. The ablation catheter of claim 1, wherein the vertices of the insert electrode converge to form a rounded distal apex configured to contact tissue.

18. The ablation catheter of claim 1 further comprising a proximal electrode coupled to the elongate body proximal of the distal electrode assembly.

19. The ablation catheter of claim 18, wherein a collective surface area of exposed portions of the distal electrode assembly is less than a surface area of an exposed portion of the proximal electrode.

20. The ablation catheter of claim 18, wherein the proximal electrode is a first proximal electrode and the ablation catheter further comprises a second proximal electrode coupled to the elongate body proximal of the first proximal electrode.

21. The ablation catheter of claim 20, wherein each of the first and second proximal electrodes are ring electrodes.

22. The ablation catheter of claim 21, wherein a ratio between a surface area of an exposed portion of the first proximal electrode and a surface area of an exposed portion of the second proximal electrode is about 1:8 to about 1:4.

22. (canceled)

23. An ablation catheter, comprising:
an elongate body having a fluid lumen extending therethrough; and
a distal electrode assembly comprising:
an insert electrode having a non-circular cross-sectional shape; and
an outer electrode positioned around the insert electrode,
wherein the insert electrode engages an inner surface of the outer electrode at a plurality of discrete contact regions spaced around a longitudinal axis of the distal electrode assembly, and
wherein interstitial spaces formed between the discrete contact regions are configured to receive fluid from the fluid lumen.

24. The ablation catheter of claim 23, wherein the insert electrode comprises an exposed length that protrudes distally beyond a distal end surface of a tip of the catheter.

25. The ablation catheter of claim 24, wherein a ratio between the exposed length of the insert electrode and a diameter of the elongate body of the catheter is about 1:8 to about 1:4.

26. The ablation catheter of claim 23, wherein a distal end surface of the outer electrode is substantially flush with a distal end surface of a tip of the catheter.

27. The ablation catheter of claim 23, wherein the outer electrode surrounds a proximal portion of the insert electrode disposed within a lumen in a tip of the catheter.

28. The ablation catheter of claim 23, wherein the insert electrode further comprises arcuate sidewalls extending between adjacent contact regions, and wherein each interstitial space is formed between one of the arcuate sidewalls and an inner surface of the outer electrode.

29. The ablation catheter of claim 1, wherein the distal electrode assembly is configured to provide laminar fluid flow through each of the interstitial spaces.

30. The ablation catheter of claim 1, wherein the insert electrode comprises a rounded distal apex.
